(19) United States
(12) Patent Application Publication
(10) Pub. No.: US 2022/0241214 A1
(43) Pub. Date: Aug. 4, 2022

(54) NANOPARTICLES COMPRISING QUINONE W METHIDES AND COMPOSITIONS FOR USE

(71) Applicant: University of Iowa Research Foundation, Iowa City, IA (US)

(72) Inventors: Aliasger K. Salem, Coralville, IA (US); Kimberly Leslie, Iowa City, IA (US); Xiangbing Meng, Iowa City, IA (US); Jianling Bi, Iowa City, IA (US); Kareem Ebeid, Iowa City, IA (US); Sudartip Areecheewakul, Iowa City, IA (US)

(21) Appl. No.: 17/620,391

(22) PCT Filed: Jun. 19, 2020
(86) PCT No.: PCT/US2020/038747

§ 371 Date: Dec. 17, 2021

Related U.S. Application Data
(60) Provisional application No. 62/864,304, filed on Jun. 20, 2019.
(60) Provisional application No. 62/865,646, filed on Jun. 24, 2019.

(51) Int. Cl. A61K 9/51; A61K 33/243; A61K 45/06

(52) U.S. Cl. CPC A61K 9/5153; B82Y 5/00; A61K 45/06; A61K 33/243

(57) ABSTRACT

A nanoparticle comprising one or more quinonemethide triterpenoids or one or more inhibitors of metadherin, and methods of using the nanoparticles, are provided.

### CROSS-REFERENCE TO RELATED APPLICATIONS

This application claims the benefit of the filing date of U.S. application No. 62/865,646, filed on Jun. 24, 2019, and U.S. application No. 62/864,304, filed on Jun. 20, 2019, the disclosures of which are incorporated by reference herein.

### STATEMENT OF GOVERNMENT RIGHTS

This invention was made with government support under R01 CA184101 and R01 CA099908 awarded by the National Institutes of Health. The government has certain rights in the invention.

### BACKGROUND

DNA damaging agents such as platinum compounds have been widely used as a primary treatment for many types of cancer including endometrial cancer, the most frequent gynecologic malignancy (Brabec & Kasparkova, 2005). However, resistance is one of the major causes of therapeutic failure for patients with metastatic or recurrent disease, thus highlighting the need to identify factors driving resistance to platinum compounds (Muggia, 2004; Galluzzi et al., 2012). Of the current genes identified on the frequently amplified region of chromosome 8, metadherin (MTDH, also known as AEG-1 and LYRIC) is a master regulator of cellular functions and is consistently associated with resistance to multiple chemotherapeutic agents, including platinum compounds (Song et al., 2015; Meng et al., 2013). Moreover, MTDH amplification is associated with metastasis and poor overall survival in multiple tumor types (Hu et al., 2009; Moelans et al., 2014). MTDH promotes cancer cell proliferation and inhibits apoptosis in part by activating classical oncogenic pathways, including Ras, myc, NFκB and PI3K/AKT (Embdad et al., 2016; Lee et al., 2006; Emdad et al., 2006).

### SUMMARY

As disclosed herein, MTDH, through its role as an RNA binding protein, regulates expression of FANCD2 and FANCI, two components of Fanconi anemia complementation group (FA) that play roles in interstrand crosslink damage induced by platinum compounds. Pristimerin, the methyl ester of celasterol, a quinonemethide triterpenoid extract from Celastraceae and Hippocrateaceae used for inflammation in traditional Chinese medicine, also found in Maytenus heterophylla, significantly decreased MTDH, FANCD2 and FANCI levels in cancer cells, thereby restoring sensitivity to platinum-based chemotherapy. Using a patient-derived xenograft model of endometrial cancer, treatment with Pristimerin in a nanoparticle formulation was found to markedly inhibit tumor growth when combined with cisplatin. These studies provide insight into the mechanism by which MTDH mediates drug resistance and identifies the pristimerin as a putative treatment to combine with antineoplastic drugs including platinum-based antineoplastic drugs. Thus, quinonemethide triterpenoids, such as those having anti-inflammatory activity which may be due to inhibition of IKK (an inhibitor of NFkappaB), optionally having chymotrypsin-like protease activity (e.g., as do some proteasome inhibitors), may be useful to increase the sensitivity of cancer cells to antineoplastic agents/drugs, e.g., drugs that cause cross-linking of DNA, thereby inhibiting DNA synthesis and/or repair. In one embodiment, the antineoplastic drug comprises a platinum drug, for example, cisplatin, carboplatin, ormaplatin (tetraplatin), oxaliplatin, DWA2114R, enloplatin, lobaplatin, CI-973 (NK-121), 254-S, JM-216, and cis-bis-neodecanoato-trans-R,R-1,2-diaminocyclohexane platinum (II). In one embodiment, the antineoplastic drug is an anthracycline, e.g., doxorubicin. In one embodiment, the antineoplastic drug is paclitaxel, tamoxifen, AZD6244 (selumetinib), 5-fluroruracil, TRAIL, an HDAC inhibitor, mitomycin C or BIBF1120 (nintedanib).

In one embodiment, the disclosure provides for a nanoparticle comprising an amount of one or more quinonemethide triterpenoids, e.g., effective to enhance sensitivity to an antineoplastic drug (enhance chemosensitivity) such as a platinum compound, or a nanoparticle comprising an amount of one or more inhibitors of metadherin, e.g., effective to enhance sensitivity to an antineoplastic drug such as a platinum compound. In one embodiment, the average diameter of a population of the nanoparticles is 200 nm, 150 nm, 125 nm, 100 nm, 75 nm, 50 nm or less. In one embodiment, the quinonemethide triterpenoid or inhibitor comprises pristimerin, a pristimerin analog, e.g., in Murayama et al., Antiviral Chem. & Chemother, 18:133 (2007), which is incorporated by reference herein, celastrol, lupeol, hydroxy-pristimerin, Tingenin B, tripterin, tripterygone, or 2-acetylphenol-1-beta-D-glucopyranosyl (1→6)-beta-D-xylpyranoside. In one embodiment the nanoparticle comprises tingenone, netzahuolcoyone, any of compounds 6-9 in Moujir et al. (Appl. Sci., 9:2957 (2019)), the disclosure of which is incorporated by reference herein, pristimerol, 8-epi-deoxoblepharodol, cel-D2 or cel-D7 in Wei et al. (Oncotarget, 5:5819 (2014)), the disclosure of which is incorporated by reference herein, any of compounds 1-11 in Zhang et al. (J. Enzyme Inhibition and Med. Chem., 33:190 (2017)), the disclosure of which is incorporated by reference herein, or compounds disclosed in WO07/077203, e.g., disclosed in Table 1 therein, the disclosure of which is incorporated by reference herein. In one embodiment, the nanoparticle comprises a polymer such as a synthetic polymer. In one embodiment, the nanoparticle comprises lactic acid, glycolic acid, or a combination thereof, e.g., a combination of lactic acid and glycolic acid. In one embodiment, a ratio of lactic acid to glycolic acid in the nanoparticles is 70:30, 75:25, 80:20, 65:35, 60:40, 55:45 or 50:50. In one embodiment, the nanoparticle comprises PEI. In one embodiment, the nanoparticle comprises a targeting ligand.

Further provided is a method to enhance inhibition of cancer by an antineoplastic agent or to enhance antineoplastic agent treatment of cancer in a mammal, e.g., a mammal having or at risk of having resistance to an antineoplastic agent. The method includes administering to the mammal a composition having an effective amount of the nanoparticle. In one embodiment, the mammal is a human. In one embodiment, the cancer is testicular cancer, ovarian cancer, lung cancer, lymphoma, bladder cancer, cervical cancer, breast cancer, esophageal cancer, colon cancer, mesothiolioma, pancreatic cancer, prostate cancer, brain cancer, neuroblastoma, or head and neck cancer. In one embodiment, the cancer is endometrial cancer, small cell lung cancer, ovarian cancer, or triple negative breast cancer. In one embodiment, the method further comprises administering an antineoplastic agent. In one embodiment, the antineoplastic agent cross links DNA, inhibits DNA repair, inhibits DNA synthesis, or a combination thereof. In one embodiment, the antineoplastic agent comprises a platinum compound. In one embodiment, the antineoplastic agent comprises cis-platin, carboplatin, oxiliplatin, nedaplatinin, picoplatin, triplatin tetranitrate, phenanthriplatin, or satraplatinin. In one embodiment, the antineoplastic agent is co-administered with the composition. In one embodiment, the antineoplastic agent is administered before or after the composition. In one embodiment, the composition is administered before or after the antineoplastic agent. In one embodiment, the composition comprises the antineoplastic agent. In one embodiment, the composition and/or the antineoplastic agent is/are locally administered. In one embodiment, the composition and/or the antineoplastic agent is/are systemically administered. In one embodiment, the composition and/or the antineoplastic agent is/are orally administered. In one embodiment, the composition and/or the antineoplastic agent is/are intravenously administered. In one embodiment, the cancer, prior to administration of the composition, is resistant to an antineoplastic agent. In one embodiment, the composition is administered to prevent development of resistance to an antineoplastic agent. In one embodiment, the mammal is resistant to the antineoplastic activity of one or more of antineoplastic drugs, e.g., a platinum drug, an anthracycline, e.g., doxorubicin, paclitaxel, tamoxifen, AZD6244, 5-fluroruracil, TRAIL, an HDAC inhibitor, mitomycin C or BIBF1120.

### BRIEF DESCRIPTION OF FIGURES

FIGS. 1A-1B. Analysis of the expression of MTDH and FANCI in endometrial and breast cancer patients. (A) MTDH amplification portends worse prognosis for endometrial cancer. Kaplan Meier analysis of MTDH copy number gain (after removal of germline copy number variants) as a measure of MTDH amplification in TCGA dataset for endometrial cancer. Blue: low MTDH copy number; red: high MTDH copy number, indicative of gene amplification. (B) MTDH amplification positively correlates with expression of FANCD2 and FANCI in breast cancer. Heat maps for MTDH copy number variations and gene expression of MTDH, FANCI, and FANCD2 in TCGA dataset for breast cancer.

FIGS. 2A-2C. FA family proteins FANCD2 and FANGI are significantly reduced in MTDH knockout mice and MTDH-depleted cancer cells. (A) Protein expression of FANCD2 and FANCI was detected by Western blotting in the brain (Br), liver (Li) and spleen (Sp) from wild type mice and two MTDH homozygous knockout mice (MTDH KO1 and MTDH KO2). PCNA, Rad51 and β-actin were also analyzed by Western blotting as controls for other DNA repair proteins and loading. (B) RT-qPCR was used to quantify the mRNA expression of MTDH, FANCD2 and FANCI in MTDH CRISPR knockout Hec50 cells relative to parental Hec50 cells. (C) FANCD2, FANCI and MTDH were detected by Western blotting in parental Hec50 cells and MTDH CRISPR knockout Hec50 cells in the absence or presence of DNA damage via cisplatin for 24 hours. Labels denote ubiquitinated (Ub) FANCI and the long (L) and short (S) isoforms of FANCD2. β-actin was used as a loading control.

FIGS. 3A-3C. Identification of the region in MTDH that associates with FANCD2 and FANCI mRNAs. (A) Schematic representation of full length MTDH and truncated MTDH constructs which contain different RNA binding domains. Circle denotes the region in MTDH that binds mRNAs (residues 145-260). (B) Immunoblot for flag-tagged MTDH fragments. Lysates were immunoprecipitated with anti-Flag, followed by Western blotting with anti-Flag antibody. (C) RNA immunoprecipitation of MTDH-associated mRNAs followed by RT-qPCR for FANCI and FANCD2. Data are presented as the fold enrichment of MTDH-bound FANCI and FANCD2 mRNA following immunoprecipitation with anti-Flag relative to IgG.

FIGS. 4A-4E. MTDH depletion increases sensitivity to cisplatin and accumulation of DNA damage in cancer cells. (A) Fluorescent imaging of MTDH (green) and nuclei (blue, DAPI) in control and MTDH CRISPR knockout cancer cells. (B) Western blot confirming deletion of MTDH using CRISPR/Cas9 in Hec50 cells. α-tubulin is the loading control. (C) Sensitivity to cisplatin was examined in parental and MTDH CRISPR knockout Hec50 cells by the WST-1 assay. ****P<0.0001 by two-way ANOVA. (D) Immunostaining was used to detect γ-H2AX foci in parental and MTDH CRISPR knockout Hec50 cells without treatment or treatment with 5 μM cisplatin for 16 hours. (E) Quantification of γ-H2AX foci in cancer cells. Data are representative of 300 cells, ***P<0.001.

FIGS. 5A-5F. Pristimerin increases cisplatin sensitivity in Hec50, MDA-MB-231 and KLE cancer cells. (A) Viability of Hec50, MDA-MB-231 and KLE cells after treatment with pristimerin for 72 hours was determined using WST-1 assay. (B-D) Viability of Hec50 (B), MDA-MB-231 (C) and KLE (D) cells after treatment with cisplatin alone or in combination with the indicated dose of pristimerin for 72 hours was determined using WST-1 assay, *P<0.05, ***P<0.001, ****P<0.0001 by 2-way ANOVA. (E) Pristimerin decreased MTDH, FANCD2 and FANCI protein levels when used as a single drug (1 μM) or in combination with cisplatin (5 μM) in Hec50, MDA-MB-231 and KLE cell lines. CT: untreated control; C+P: cisplatin+pristimerin. (F) Quantification of Western blots in (E). Data are the average of 3 independent experiments. *P<0.05, **P<0.01,***P<0.001 vs. control by Student's t-test.

FIGS. 6A-6C. Pristimerin-loaded nanoparticles reduce the expression of MTDH, FANCD2 and FANCI in Hec50, MDA-MB-231 and KLE cells (A) SEM images of pristimerin-loaded PLGA particles. (B) Comparison of the effect of pristimerin in solution and loaded into nanoparticles (NP) on expression of MTDH, FANCD2 and FANCI and induction of the apoptotic marker cleaved caspase 3 and the autophagy marker LC3 in Hec50, MDA-MB-231 and KLE cells. β-actin: loading control; CT: untreated. (C) Quantification of Western blots in (B). With the exception of cleaved caspase 3 and LC3B, all data are relative to control. For cleaved caspase 3 and LC3B data are relative to pristimerin in solution. Data are the average of 3 independent experiments. *P<0.05, **P<0.01, ***P<0.001 vs. control.

FIGS. 7A-7C. Nanoparticle-delivered pristimerin increases cisplatin sensitivity in a PDX model of endometrial cancer. (A) Growth curves for tumor volumes in PDX1 mice. Treatment began on day 15 post-implantation of PDX1 and continued for 4 weeks. Pristimerin (NP): nanoparticle-loaded pristimerin, *P<0.05, **P<0.01, ****P<0.0001 by 2-way ANOVA. (B) Tumor weight was determined at the completion of treatment. *P<0.05, **P<0.01, ****P<0.0001 vs. control by Student's t-test. (C) Images of tumor size at the completion of treatment. Note that pristimerin+cisplatin caused complete tumor regression in 3 of the 5 mice.

FIG. 8. Celastrol can reduce MTDH and FANCI protein levels in cancer cells.

FIG. 9. Histogram of particle size.

FIG. 10. Protein levels of the endoplasmic reticulum (ER) stress biomarker CHOP, the apoptosis biomarker cleaved caspase 3 and the autophagy biomarker LC3B were all increased by treatment of Hec50, MDA-MB-231 and KLE cells with pristimerin-loaded nanoparticles.

FIG. 11. High expression of MTDH with levels similar to those observed in Hec50 cells.

FIG. 12. Effect on mice's body weights by treatments of pristimerin, cisplatin alone or in combination.

FIGS. 13A-13F. FA family proteins FANCD2 and FANCI are significantly reduced in MTDH knockout mice and MTDH-depleted cancer cells and increased in MTDH overexpressed cancer cells. (A) Expression of FANCD2 and FANCI protein was detected and quantified by Western blotting in the brain (Br), liver (Li) and spleen (Sp) from wild type mice and two MTDH homozygous knockout mice (MTDH KO1 and MTDH KO2). Rad51 and β-actin were also analyzed by Western blotting as controls for other DNA repair proteins and loading. (B) Quantification of Western blots in (A). With the exception of Rad51, all data are relative to wide type brain. For Rad51 data are relative to MTDH-′-2 spleen. (C, D) FANCD2, FANCI and MTDH were detected and quantified by Western blotting in parental Hec50 cells and MTDH CRISPR knockout Hec50 cells in the absence or presence of DNA damage via cisplatin for 24 hours. Labels denote ubiquitinated (Ub) FANCI and the long (L) and short (S) isoforms of FANCD2. β-Actin was used as a loading control. (E, F) FANCD2, FANCI and MTDH were detected and quantified by Western blotting in empty vector transfected Hec50 cells and MTDH overexpressed Hec50 cells. β-Actin was used as a loading control. Each figure is representative of three independent experiments.

FIGS. 14A-14B. Analysis of the expression of MTDH and FANCI in endometrial and breast cancer patients. (A) MTDH amplification portends worse prognosis for endometrial cancer. Kaplan Meier analysis of MTDH copy number gain (after removal of germline copy number variants) as a measure of MTDH amplification in TCGA dataset for endometrial cancer. Blue: low MTDH copy number; red: high MTDH copy number, indicative of gene amplification. (B) MTDH amplification positively correlates with expression of FANCD2 and FANCI in breast cancer. Heat maps for MTDH copy number variations and gene expression of MTDH, FANCI, and FANCD2 in TCGA dataset for breast cancer.

FIGS. 15A-15C. Identification of the region in MTDH that associates with FANCD2 and FANCI mRNAs. (A) Schematic representation of full length MTDH and truncated MTDH constructs which contain different RNA binding domains. Circle denotes the region in MTDH that binds mRNAs (residues 145-260). (B) Immunoblot for flag-tagged MTDH fragments. Lysates were immunoprecipitated with anti-Flag, followed by Western blotting with anti-Flag antibody. (C) RNA immunoprecipatation of MTDH-associated mRNAs followed by RT-qPCR for FANCI and FANCD2. Data are presented as the fold enrichment of MTDH-bound FANCI and FANCD2 mRNA following immunoprecipitation with anti-Flag relative to IgG. Error bars represent the relative rate of enrichment of FANCD2 and FANCI from three independent experiments, P<0.01.

FIGS. 16A-16E. MTDH depletion increases sensitivity to cisplatin and accumulation of DNA damage in cancer cells. (A) Fluorescent imaging of MTDH (green) and nuclei (blue, DAPI) in control and MTDH CRISPR knockout cancer cells. (B) Western blot confirming deletion of MTDH using CRISPR/Cas9 in Hec50 cells. α-Tubulin is the loading control. (C) Sensitivity to cisplatin was examined in parental and MTDH CRISPR knockout Hec50 cells by the WST-1 assay from three independent experiments. ****P<0.0001 by two-way ANOVA. (D) Immunostaining was used to detect γ-H2AX foci in parental and MTDH CRISPR knockout Hec50 cells without treatment or treatment with 5 μM cisplatin for 16 hours. (E) Quantification of γ-H2AX foci in cancer cells. Data are representative of 300 cells from three independent experiments, ***P<0.001. (For interpretation of the references to color in this figure legend, the reader is referred to the web version of this article.)

FIGS. 17A-17H. Pristimerin increases cisplatin sensitivity in Hec50, MDA-MB-231 and KLE cancer cells. (A) Viability of Hec50, MDA-MB-231 and KLE cells after treatment with pristimerin for 72 hours was determined using WST-1 assay. (BeD) Viability of Hec50 (B), MDA-MB-231 (C) and KLE (D) cells after treatment with cisplatin alone or in combination with the indicated dose of pristimerin for 72 hours was determined using WST-1 assay, *P<0.05, ***P<0.001, ****P<0.0001 by 2-way ANOVA. (E) Pristimerin decreased MTDH, FANCD2 and FANCI protein levels when used as a single drug (1 μM) or in combination with cisplatin (5 μM) in Hec50, MDA-MB-231 and KLE cell lines. CT: untreated control; C+P: cisplatin+pristimerin. (F) Comparison of the effect of pristimerin in solution (1 μM) and loaded into nanoparticles (NP) (40 mM) on expression of MTDH, FANCD2 and FANCI and induction of the apoptotic marker cleaved caspase 3 and the autophagy marker LC3 in Hec50, MDA-MB-231 and KLE cells. β-Actin: loading control; CT: untreated. (G) Quantification of Western blots in (E). (H) Quantification of Western blots in (F) With the exception of cleaved caspase 3 and LC3B, all data are relative to control. For cleaved caspase 3 and LC3B data are relative to pristimerin in solution. All data are representative of three independent experiments.

FIGS. 18A-18C. Nanoparticle-delivered pristimerin increases cisplatin sensitivity in a PDX model of endometrial cancer. (A) Growth curves for tumor volumes in PDX1 mice. Treatment began on day 15 post-implantation of PDX1 and continued for 4 weeks. Pristimerin (NP): nanoparticle-loaded pristimerin, *P<0.05, **P<0.01, ****P<0.0001 by 2-way ANOVA. (B) Tumor weight was determined at the completion of treatment. *P<0.05, **P<0.01, ****P<0.0001 vs. control by Student's t-test. (C) Images of tumor size at the completion of treatment. Note that pristimerin+cisplatin caused complete tumor regression in 3 of the 5 mice.

FIGS. 19A-19B. MTDH deletion and MTDH overexpression had no effect on the mRNA levels of FANCD2 and FANCI. (A) RT-qPCR was used to quantify the mRNA expression of MTDH, FANCD2 and FANCI in MTDH CRISPR knockout Hec50 cells relative to scrambled sgRNA transfected Hec50 cells. (B) RT-qPCR was used to quantify the mRNA expression of MTDH, FANCD2 and FANCI in MTDH overexpressed Hec50 cells relative to empty vector transfected Hec50 cells.

FIG. 20. Cell proliferation of cancer cells expressing scrambled sgRNA and multiple MTDH knockout clones were detected with XTT assay.

FIG. 21. Celastrol reduced protein levels of MTDH, FANCD2 and FANCI in Hec50 cells after 24 hours treatment.

FIG. 22. Overexpression of MTDH did not protect from pristimerin-induced cell death.

FIG. 23. Cell proliferation and sensitivity to pristimerin between cancer cells expressing scrambled sgRNA and cancer cells with multiple MTDH knockout clones were detected.

FIGS. 24A-24C. Histogram of particle size (A) and SEM images of pristimerin-loaded PLGA particles (B).

FIG. 25. Pristimerin (in solution) induced expression of ER stress marker CHOP after 16 hours treatment (1 μM) or in combination with cisplatin (5 μM). CT: control untreated; C+P: cisplatin+pristimerin; β-actin: loading control.

FIG. 26. MTDH expression in endometrial cancer cells and PDX mouse models was detected by western blotting. α-Tubulin was the loading control. (Ishikawa cell: endometrial cancer cell line)

FIG. 27. Body weights by treatments of pristimerin, cisplatin, and combination of pristimerin and cisplatin.

### DETAILED DESCRIPTION

MTDH was reported to be an RNA binding protein (Meng et al., 2012) The association of MTDH with mRNAs has the potential to regulate drug resistance by controlling post-transcriptional processing of multiple proteins. Indeed, two independent studies revealed that MTDH binds to mRNAs that encode several Fanconi anemia (FA) pathway proteins (Meng et al., 2012; Hsu et al., 2018). The FA pathway plays a critical role in DNA repair following interstrand crosslink damage induced by platinum compounds (Nakanishi 2005). Studies in patients with mutations in FA pathway proteins demonstrate that, while there is increased risk for cancer development, tumors harboring these mutations respond well to chemotherapy (van der Heijden et al., 2003; Ceccaldi et al., 2016). It was hypothesized that downregulation of the FA pathway by targeting MTDH has the potential to increase sensitivity to platinum-based DNA damaging agents. The objective of this study was to elucidate the role of MTDH on FA pathway regulation and resistance to platinum compounds.

Metadherin (MTDH, also known as AEG-1 and LYRIC), located at frequently amplified region of chromosome 8, has been consistently associated with resistance to chemotherapeutic agents, though the precise mechanisms remain incompletely defined. Herein compelling evidence is provided that MTDH, through its role as an RNA binding protein, regulates expression of FANCD2 and FANCI, two components of the Fanconi anemia complementation group (FA) that play critical roles in interstrand crosslink damage induced by platinum compounds. Pristimerin, a quinonemethide triterpenoid extract from Celastraceae and Hippocrateaceae used for inflammation in traditional Chinese medicine, significantly decreased MTDH, FANCD2 and FANCI levels in cancer cells, thereby restoring sensitivity to platinum-based chemotherapy. Using a patient-derived xenograft model of endometrial cancer, it was discovered that treatment with pristimerin in a nanoparticle formulation markedly inhibited tumor growth when combined with cisplatin. These studies provide insight into the mechanism by which MTDH mediates drug resistance and identifies the natural agent pristimerin as a putative treatment to combine with platinum-based chemotherapeutic drugs for tumors with amplification or excessive expression of MTDH.

### Exemplary Delivery Vehicles

Delivery vehicles for a MDTH inhibitor or quinonemethide triterpenoid include, for example, naturally occurring or synthetic polymers that form microparticles, nanoparticles, or other macromolecular complexes capable of mediating delivery of a MDTH inhibitor or quinonemethide triterpenoid. Vehicles can also comprise other components or functionalities that further modulate, or that otherwise provide beneficial properties.

In one embodiment, the delivery vehicle is a naturally occurring polymer, e.g., formed of materials including but not limited to albumin, collagen, fibrin, alginate, extracellular matrix (ECM), e.g., xenogeneic ECM, hyaluronan (hyaluronic acid), chitosan, gelatin, keratin, potato starch hydrolyzed for use in electrophoresis, or agar-agar (agarose). In one embodiment, the delivery vehicle comprises a hydrogel. In one embodiment, the composition comprises a naturally occurring polymer. For example, the MDTH inhibitor or quinonemethide triterpenoid may be in nanoparticles or microparticles. Table 1 provides exemplary materials for delivery vehicles that are formed of naturally occurring polymers and materials for particles.

TABLE 1

| Particle class | Materials |
|---|---|
| Natural materials or | Chitosan |
| derivatives | Dextran |
| | Gelatine |
| | Albumin |
| | Alginates |
| Polymer carriers | Liposomes |
| | Starch |
| | Polylactic acid |
| | Poly(cyano)acrylates |
| | Polyethyleneimine |
| | Block copolymers |
| | Polycaprolactone |

An exemplary polycaprolactone is methoxy poly(ethylene glycol)/poly(epsilon caprolactone). An exemplary poly lactic acid is poly(D,L-lactic-co-glycolic)acid (PLGA).

Some examples of materials for particle formation include but are not limited to agar acrylic polymers, polyacrylic acid, poly acryl methacrylate, gelatin, poly(lactic acid), pectin (poly glycolic acid), cellulose derivatives, cellulose acetate phthalate, nitrate, ethyl cellulose, hydroxyl ethyl cellulose, hydroxypropylcellulose, hydroxyl propyl methyl cellulose, hydroxypropylmethylcellulose phthalate, methyl cellulose, sodium carboxymethylcellulose, poly(ortho esters), polyurethanes, poly(ethylene glycol), poly(ethylene vinyl acetate), polydimethylsiloxane, poly(vinyl acetate phthalate), polyvinyl alcohol, polyvinyl pyrrollidone, and shellac. Soluble starch and its derivatives for particle preparation include amylodextrin, amylopectin and carboxy methyl starch.

In one embodiment, the polymers in the nanoparticles or microparticles are biodegradable. Examples of biodegradable polymers useful in particles preparation include synthetic polymers, e.g., polyesters, poly(ortho esters), polyanhydrides, or polyphosphazenes; natural polymers including proteins (e.g., collagen, gelatin, and albumin), or polysaccharides (e.g., starch, dextran, hyaluronic acid, and chitosan). For instance, a biocompatible polymer includes poly (lactic) acid (PLA), poly (glycolic acid) (PLGA). Natural polymers that may be employed in particles (or as the delivery vehicle) include but are not limited to albumin, chitin, starch, collagen, chitosan, dextrin, gelatin, hyaluronic acid, dextran, fibrinogen, alginic acid, casein, fibrin, and polyanhydrides.

In one embodiment, the delivery vehicle is a hydrogel. Hydrogels can be classified as those with chemically crosslinked networks having permanent junctions or those with physical networks having transient junctions arising from polymer chain entanglements or physical interactions, e.g., ionic interactions, hydrogen bonds or hydrophobic interactions. Natural materials useful in hydrogels include natural polymers, which are biocompatible, biodegradable, support cellular activities, and include proteins like fibrin, collagen and gelatin, and polysaccharides like starch, alginate and agarose.

In one embodiment, the delivery vehicle comprises inorganic nanoparticles, e.g., calcium phosphate or silica particles; polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines. A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N-(N,N-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed. In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, the particles comprise at least one polymeric material. In one embodiment, the polymeric material is biodegradable. In one embodiment, polymeric materials include: silk, elastin, chitin, chitosan, poly(α-hydroxy acids), poly(anhydrides), and poly(orthoesters). In one embodiment, the biodegradable microparticle may comprise polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo) phosphazenes], poly-hydroxybutyric acid, or S-caproic acid, polylactide-co-glycolide, polylactic acid, and polyethylene glycol. Polyesters may be employed. In one embodiment, PLGA is employed, e.g., PLGA 75:25, PLGA 50:50 and PLGA 85:15.

### Formulations and Dosages

The nanoparticles having a MDTH inhibitor or quinonemethide triterpenoid can be formulated as pharmaceutical compositions and administered to a mammalian host, such as a human patient in a variety of forms adapted to the chosen route of administration, e.g., orally or parenterally, by intravenous, intramuscular, topical, local, or subcutaneous routes. In one embodiment, the composition having isolated polypeptide or peptide is administered to a site of bone loss or cartilage damage or is administered prophylactically.

In one embodiment, the nanoparticles may be administered by infusion or injection. Solutions of the MDTH inhibitor or quinonemethide triterpenoid or its salts, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in complexes, liposomes, nanoparticles or microparticles. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In some cases, it may be preferable to include isotonic agents, for example, sugars, buffers or sodium chloride. Prolonged absorption of the injectable compositions can be brought about by the use in the compositions of agents delaying absorption, for example, microparticles, or aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the MDTH inhibitor or quinonemethide triterpenoid can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the MDTH inhibitor or quinonemethide triterpenoid in a composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the MDTH inhibitor or quinonemethide triterpenoid for use alone or with other agents will vary with the route of administration, the nature of the condition being treated and the age and condition of the patient and will be ultimately at the discretion of the attendant physician or clinician.

The MDTH inhibitor or quinonemethide triterpenoid in the nanoparticles may be conveniently administered in unit dosage form; for example, containing 5 to 1000 mg, conveniently 10 to 750 mg, or conveniently 50 to 500 mg of active ingredient per unit dosage form.

In general, a suitable dose may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

### Exemplary Particle Sizes (Diameters)

In one embodiment, the particle is a nanoparticle. In one embodiment, the particle may be about 50 nm to less than about 1000 nm, about 100 nm to about 900 nm, about 400 nm to about 800 nm, or about 500 nm to about 700 nm, in diameter. In various aspects, the nanoparticles which range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm. The size of the nanoparticles may be from about 5 nm to about 150 nm (mean diameter), from about 30 to about 100 nm, from about 40 to about 80 nm. The size of the nanoparticles may be from about 25 nm to about 200 nm (mean diameter), from about 30 to about 150 nm, from about 50 to about 100 nm, or from about 75 nm to about 125 nm.

Microparticles, in contrast to nanoparticles, for use in the composition of the invention are 1.0 μm up to about 100 μm, and in one embodiment up to about 3.0 μm.

### EXEMPLARY EMBODIMENTS

In one embodiment, nanoparticles comprising an amount of one or more quinonemethide triterpenoids or an amount of one or more inhibitors of metadherin is provided. In one embodiment, a composition comprises the nanoparticles in an amount that enhances sensitivity to an anti-neoplastic compound, e.g., a platinum compound. In one embodiment, the diameter of the nanoparticle is 200 nm or less. In one embodiment, the quinonemethide triterpenoid or the inhibitor comprises pristimerin, celastrol, lupeol, hydroxy-pristimerin, Tingenin B, tripterin, tripterygone, or 2-acetylphenol-1-beta-D-glucopyranosyl (1-->6)-beta-D-xylpyranoside. In one embodiment, the nanoparticle has a diameter of about 25 nm to about 200 nm. In one embodiment, the nanoparticle comprises a synthetic polymer. In one embodiment, the polymer comprises lactic acid, glycolic acid, caproic acid, a polyanhydride, or a combination thereof. In one embodiment, the polymer comprises lactic acid and glycolic acid, polycaprolactone or polylactic acid. In one embodiment, the polymer comprises a ratio of lactic acid to glycolic acid of 70:30, 75:25, 80:20, 65:35, 60:40, 55:45 or 50:50. In one embodiment, the polymer is a polyaziridine. In one embodiment, the polymer comprises polyethylenimine (PEI). In one embodiment, the nanoparticle further comprises a targeting ligand.

In one embodiment, a method to decrease resistance to, or enhance sensitivity to, an antineoplastic agent in a mammal having cancer is provided. The method includes: administering to the mammal an effective amount of a composition having the nanoparticles. In one embodiment, the mammal is a human. In one embodiment, the cancer is testicular cancer, ovarian cancer, lung cancer, lymphoma, bladder cancer, cervical cancer, breast cancer, esophageal cancer, colon cancer, mesothelioma, pancreatic cancer, prostate cancer, brain cancer, neuroblastoma, or head and neck cancer. In one embodiment, the cancer is endometrial cancer, small cell lung cancer, ovarian cancer, or triple negative breast cancer. In one embodiment, the method further includes administering an antineoplastic agent, e.g., one that cross links DNA, inhibits DNA repair, inhibits DNA synthesis, or a combination thereof. In one embodiment, the agent is a platinum compound, e.g., cis-platin, carboplatin, oxiliplatin, nedaplatinin, picoplatin, triplatin tetranitrate, phenanthriplatin, or satraplatinin. In one embodiment, the antineoplastic agent is co-administered with the composition.

In one embodiment, the antineoplastic agent is administered before or after the composition, or both. In one embodiment, the composition comprises the antineoplastic agent. In one embodiment, the composition is locally administered. In one embodiment, the composition is systemically administered.

In one embodiment, the composition is orally administered. In one embodiment, the composition is intravenously administered. In one embodiment, the cancer, prior to administration of the composition, is resistant to an antineoplastic agent. In one embodiment, prior to administration of the composition, the cancer is resistant to an antineoplastic agent that cross links DNA, inhibits DNA repair, inhibits DNA synthesis, or a combination thereof. In one embodiment, prior to administration of the composition, the cancer is resistant to a platinum compound. In one embodiment, the anti-neoplastic agent is systemically administered. In one embodiment, the anti-neoplastic agent is injected. In one embodiment, the anti-neoplastic agent is intravenously administered.

In one embodiment, a mammal has a cancer that is or is suspected of being resistant to an anti-neoplastic agent. The mammal is administered the nanoparticles disclosed herein in an amount that enhances the activity of the anti-neoplastic agent. The nanoparticles may be administered before, during or after, or any combination thereof, administration of the anti-neoplastic agent.

The invention will be further described by the following non-limiting examples.

### Example 1

### Material and Methods

### Analysis of MTDH, FANCD2 and FANCI in TCGA Data

UCSC Xena (https://xena.ucsc.edu) is a public hub that was used to analyze the correlation of MTDH amplification with MTDH, FANCD2 and FANCI expression in endometrial cancer and breast cancer patients in TCGA (The Cancer Genome Atlas). Gene expression was determined by RNA-sequencing in the TCGA dataset. MTDH amplification was determined by analyzing copy number variation (CNV) for MTDH after removal of germine CNVs for MTDH.

### Cell Line and Culture Conditions

Hec50 uterine serous carcinoma cells were kindly provided by Dr. Erlio Gurpide in 1991 (New York University) (Granvanis et al., 1986). KLE uterine serous carcinoma cells and MDA-MB-231 breast cancer cells were purchased from American Type Culture Collection in 2009 (ATCC, Manassas, Va.). Hec50 and MDA-MB-231 cells were cultured in Dulbecco's modified Eagle's medium (DMEM) supplemented with 10% fetal bovine serum (FBS, Gibco, Grand Island, N.Y.) with penicillin/streptomycin. KLE cells were cultured in RPMI-1640 (Gibco) supplemented with 10% FBS with penicillin/streptomycin. Cell line authentication was performed yearly for all studied lines using the CODIS marker testing. Mycoplasma testing was performed annually by the University of Iowa DNA Sequencing Core facility. Cells were used over no more than 10 passages from thawing to the completion of all experiments.

### Western Blotting

Cells were scraped into ice-cold RIPA buffer (50 mM Tris-HCl pH 7.4, 150 mM NaCl, 2 mM EDTA, 1% NP-40, 0.1% SDS with protease inhibitor) and sonicated three times. Lysates were then centrifuged at 12000×g for 15 minutes at 4° C. and protein was quantified by BCA assay (Thermo Fisher Scientific, Waltham, Mass., USA). Samples were separated on 10% SDS-PAGE gels then transferred to a nitrocellulose blotting membrane, which was blocked with 5% nonfat milk and incubated overnight at 4° C. with primary antibodies. Anti-FANCD2 (1:2000, #NB 200-182) and anti-FANCI (1:500, #ab74332) were obtained from Novus Biologicals (Centennial, Colo., USA). Anti-PCNA (1:1000, #13110), anti-RAD51 (1:1000, #8875), anti-LC3B (1:1000, #3868) and anti-cleaved caspase 3 (1:1000, #9661) were from Cell Signaling Technology (Danvers, Mass.). Anti-β-actin (1:10000, #A5441) was from Sigma, St. Louis, Mo. Anti-MTDH (1:250, #517220) was from Santa Cruz Biotechnology, Dallas, Tex. Membranes were further incubated with appropriate secondary antibodies (1:10000, #7076 and #7074, Cell Signaling Technology) at room temperature for 2 hours. Protein bands were detected using the Bio-Rad ChemiDoc system, and densitometry was analyzed with BioRad Image Lab Software (Bio-Rad Laboratories, Hercules, Calif.).

### Cell Viability Assays

Cell viability was determined by WST-1 assay. Cells were seeded into 96-well plates (1×10⁴ cells per well) then treated with cisplatin (Fresenius Kabi Oncology Ltd, Haryana, India) or the combination of cisplatin with pristimerin in solution (Cayman Chemical, Ann Arbor, Mich.). Cell viability was evaluated using the cell proliferation reagent WST-1 (Roche, Germany) according to the manufacturer's protocol. The absorbance was measured with a micro-plate reader (BioRad). Data were calculated as percent (%) viability relative to untreated control, which was set at 100%.

### Immunofluorescence (IF) Staining

Hec50 cells were seeded on coverslips then fixed with 2% paraformaldehyde for 20 minutes. Coverslips were rinsed 3 times with 1 ml PBS and incubated with 80% ice-cold methanol for 1 hour, followed by permeabilization for 25-30 minutes with 0.2% Triton X-100. Cells were blocked with 3% BSA then incubated with specific antibodies at 4° C. overnight. Anti-MTDH (1:100, #14065), anti-phospho-histone H2AX (Ser139) (1:400, #9718) were from Cell Signaling Technology (Danvers, Mass.). Then, cells were incubated with Alexa Fluor 546-conjugated anti-rabbit secondary antibody (1:200, Cell Signaling Technology) at room temperature for 2 hours; nuclei were stained using mounting solution with DAPI (Vector Laboratories). Visualization was performed on a Zeiss 710 confocal microscope.

### Animal Studies

All animal studies were performed under animal protocols #7051085 approved by the University of Iowa Institutional Animal Care and Use Committee (Iowa City, Iowa).

MTDH knockout mice: MTDH knockout mice were generated as described previously (Meng et al., 2015). Male mice at 20 weeks of age were euthanized and the spleen, brain and liver were removed and immediately snap frozen in liquid nitrogen. Tissue was ground to a fine powder in liquid nitrogen and then protein was extracted for Western blotting.

Patient-derived xenograft (PDX) studies: A PDX model of endometrial cancer (PDX1) has been previously described (Luo et al., 2010). NOD.Cg-Prkdcscid Il2rgtm1Wjl/SzJ (NSG, Jackson Laboratories, Bar Harbor, Me.) immunodeficient female mice at 8 weeks of age were injected with passage 3 of PDX1 tumor tissue (10 mg/100 μl medium) into the right flank subcutaneously. The mice were randomly divided into 4 groups, with 5 mice per drug treated group, and 4 mice comprised the empty nanoparticle control group. Treatment was started on day 15 after engraftment of cells. The dose of pristimerin delivered in nanoparticles was 60 μg for each mouse and was administered by intravenous injection (IV) injection twice a week for a total of 4 weeks. The dose of cisplatin was 500 mg for each mouse and was administered by intraperitoneal (IP) injection twice a week for a total of 4 weeks. After 2 weeks of treatment, tumors were measured weekly using calipers, and volumes were calculated using the formula length×width²/2.

### MTDH Silencing by CRISPR Editing

The knockout of MTDH expression using CRISPR/Cas9 was achieved as described previously (Kavlashvili et al., 2016). The sgRNA CAAAACAGTTCACGCCATGA (SEQ ID NO:1) targeted the coding region of the MTDH gene at 97686713 to 97686733 (Sequence ID: NC_000008.11 at Homo sapiens chromosome 8, GRCh38.p12). The sgRNA was cloned into lentiCRISPRv1 (Addgene Plasmid 49535, Addgene, Watertown, Mass., USA). The viral vectors were produced in HEK293T cells following the manufacturer's protocol. Cells were infected with the lentivirus and cultured in the presence of puromycin. Single cell clones were selected by limiting dilution. MTDH deletion was confirmed by qPCR and by Western blotting.

### Preparation of Pristimerin-Loaded Nanoparticles

Pristimerin-loaded Poly (DL-lactide-co-glycolide) (PLGA) nanoparticles were prepared using the nanoprecipitation method as described previously (Ebeid et al., 2018). Briefly, 2 mg of pristimerin and 20 mg of 75:25 Poly (DL-lactide-co-glycolide) (Lactel Absorbable Polymers, Birmingham, Ala.) were dissolved in 3.4 ml of acetone, sonicated for 10 minutes (Branson® 5200), and then mixed with 0.6 ml of 97% ethanol. This organic solution was added drop wise into a stirred aqueous solution prepared by mixing 20 ml distilled water with 0.6 ml of 1% (w/v) D-α-Tocopherol polyethylene glycol 1000 succinate (Sigma Aldrich). The organic solvent in the nanoparticle suspension was evaporated under reduced pressure of 50 mBar for 6 hours using a rotary evaporator (Heidolph, Laborota 4000-efficient). Nanoparticles were then washed 4 times using Amicon ultra-15 centrifugal filter units (MW cutoff=100 kDa (EMD Millipore)) by centrifugation at 500 g for 20 minutes (Eppendorf® centrifuge 5804 R). Pristimerin-loaded nanoparticles were freshly prepared before each experiment.

### Quantification of Pristimerin Loading

In order to determine pristimerin loading per mg of nanoparticles, freshly prepared pristimerin-loaded nanoparticles were frozen overnight and then lyophilized using a Labconco freeze dryer (FreeZone 4.5). Known amounts of lyophilized pristimerin-loaded nanoparticles were dissolved in acetonitrile, and then pristimerin loading was quantified using high performance liquid chromatography (HLPC, Waters, 2690 separations module) equipped with an ultraviolet detector (Waters, 2487 Dual λ absorbance detector) using 425 nm as the detection wavelength. The column was a Symmetry Shield™ RP 18, 5 μm, 4.6×150 mm. Isocratic elution was carried out using a mobile phase consisting of a mixture of methanol and ultrapure water+0.1% (v/v) phosphoric acid (80:20) at a flow rate of 1 ml/minutes with 10 μl as the injection volume. A standard curve of known concentrations of pristimerin solution in acetonitrile was generated and used to determine pristimerin loading in the nanoparticles.

Drug loading and encapsulation efficiency (% EE) were calculated from equations 1 and 2, respectively. In the equations, nanoparticles are abbreviated as “NPs.”

$$\begin{matrix}Drug loading ( \frac{μg of drug}{mg of NPs} ) = \frac{Amount of pristimerin in NPs ( μg )}{Total weight of NPs ( mg )} & ( 1 ) \\ Encapsulation efficiency ( \% ) = \frac{Amount of pristimerin in NPs ( mg )}{Initial amount of pristimerin ( mg ) } \times 100 & ( 2 )\end{matrix}$$

### Pulldown of MTDH-Associated RNAs

Magna RIP™ (RNA-binding protein immunoprecipitation) kit (Millipore, Bedford, Mass.) and real time PCR were used to pull down MTDH-associated RNAs and to identify mRNAs that associate with MTDH per the manufacturer's protocol as previously described (Meng et al., 2012). Anti-MTDH (40-6500, 5 μg/l ml, ThermoFisher, Inc., Waltham, Mass.) was used to pull down MTDH-associated mRNAs, and anti-IgG (5 μg/1 ml, Millipore, Bedford, Mass.) was used as a negative control.

### Statistical Analysis

Kaplan Meier analysis was used to determine the association of MTDH amplification with survival in endometrial cancer TCGA dataset. Two-sided paired t-tests were used to compare test sets with controls. Two-way ANOVA was used for comparisons between control and treatment over a range of doses or times. P values are denoted as follows: “*” ≤0.05, “*” ≤0.01, “***” ≤0.01, “****” ≤0.0001.

### Results

### Analysis of the Expression of MTDH and FANCI in Endometrial and Breast Cancer Patients

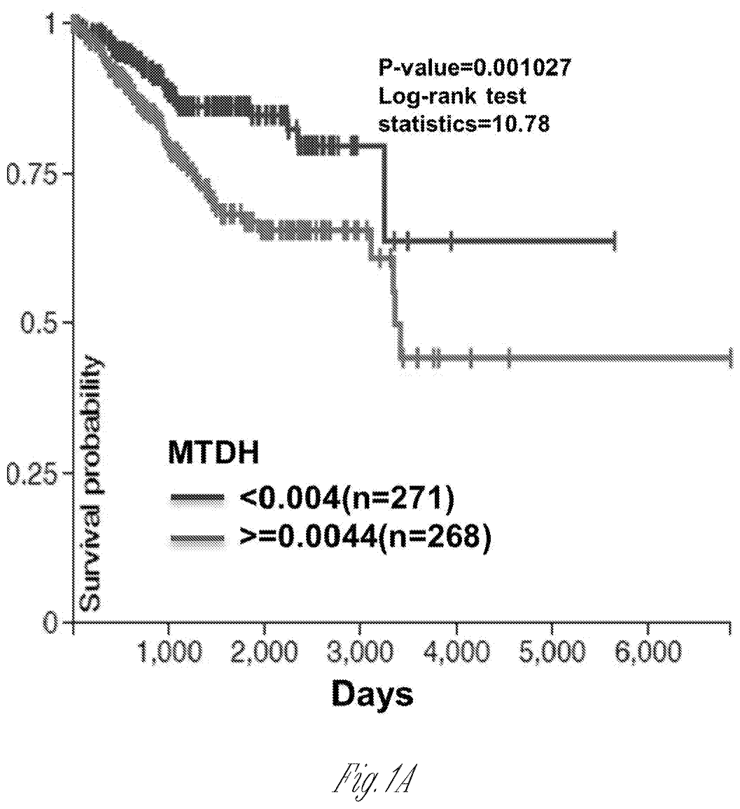

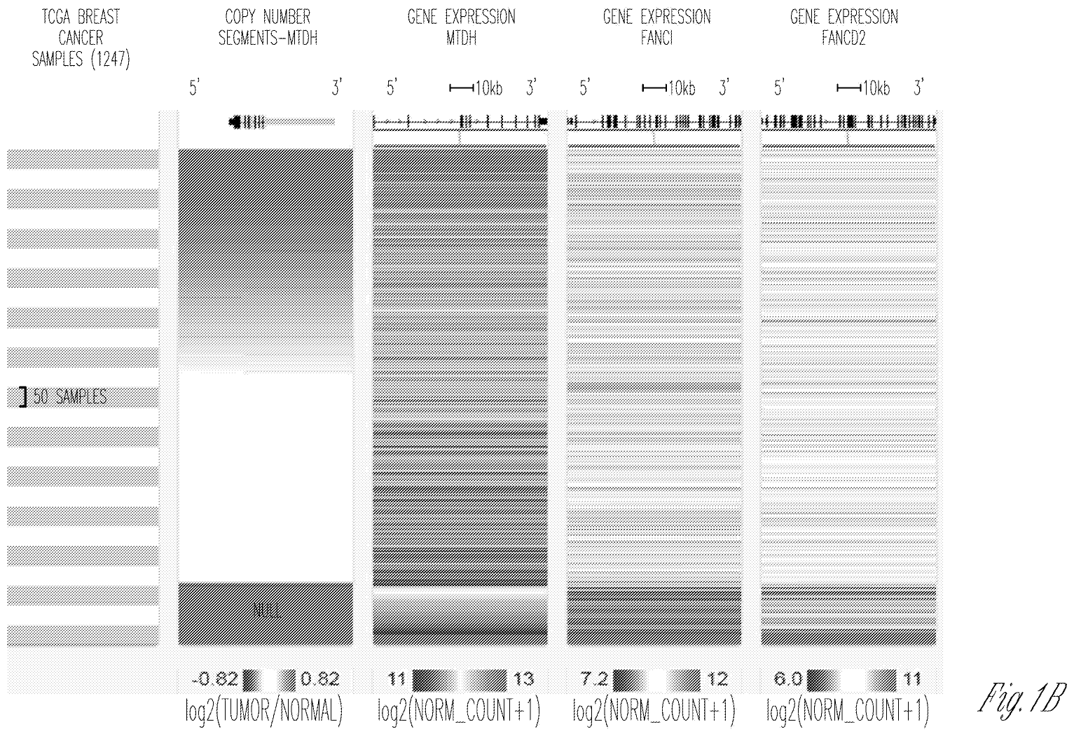

MTDH amplification negatively correlates with overall survival in breast cancer patients (Hu et al., 2009). Using TCGA dataset for endometrial cancer, it was substantiated that MTDH amplification is also associated with poor survival in endometrial cancer (FIG. 1A). Amplification and increased expression of MTDH also positively correlated with the expression of FANCI and FANCD2 in TCGA dataset for breast cancer (FIG. 1B).

### MTDH Depletion Causes a Reduction in FANCD2 and FANCI Proteins

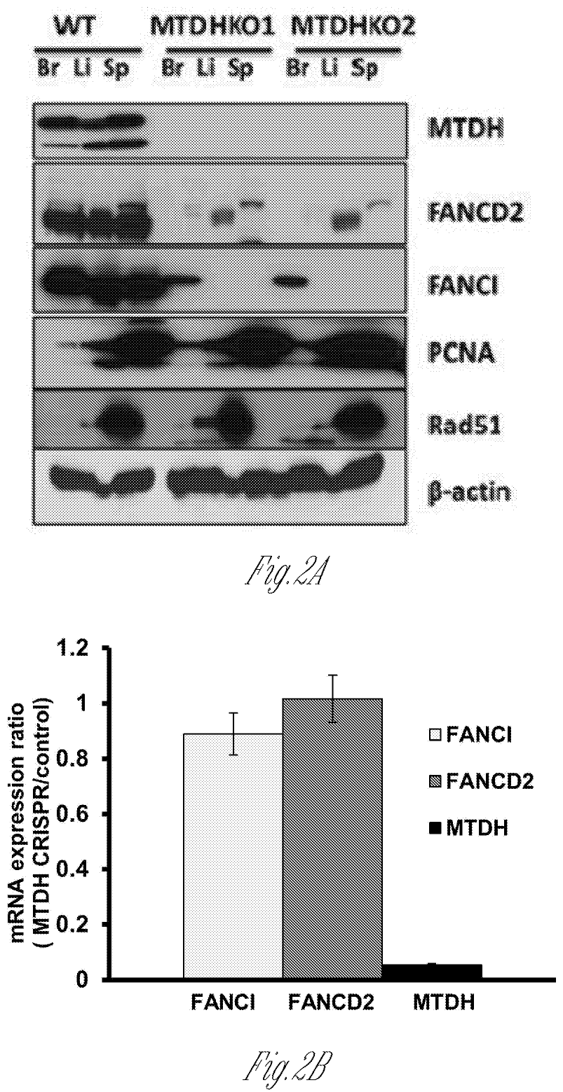

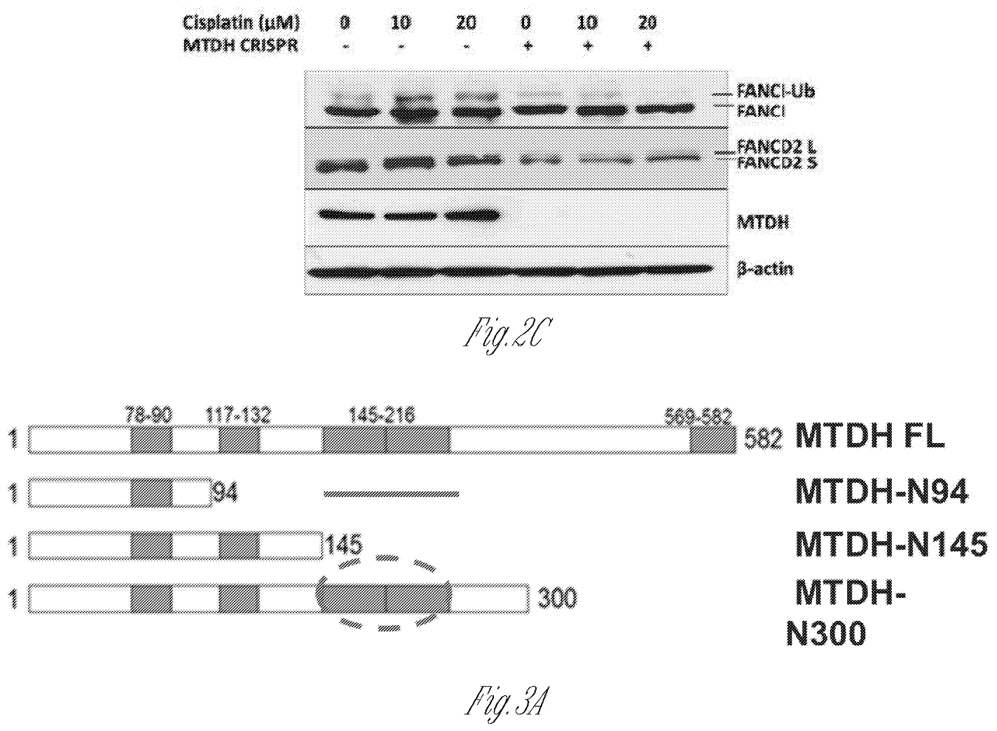

Previous work established that MTDH binds with mRNAs corresponding to FANCD2 and FANCI proteins (Meng et al., 2012). This observation was recently validated by deep sequencing of MTDH-associated transcripts, which were precipitated by an anti-MTDH antibody after protein and mRNA crosslinking (Table 2) (Hsu et al., 2018). Of note, Hsu, Jack C-C et al demonstrated that MTDH binds to several regions within the FANCI and FANCD2 mRNA sequences. To determine whether MTDH contributes to changes in FANCD2 and FANCI expression at the protein level, the expression of FANCD2, FANCI and other DNA repair proteins was examined in tissues from MTDH knockout mice, which were generated by homozygous deletion of exon 3 in the Mtdh gene (Hu et al., 2009). A dramatic reduction of FANCD2 and FANCI was detected in the liver, brain and spleen from MTDH knockout mice, though expression of other DNA repair proteins such as PCNA and Rad51 remained unchanged (FIG. 2A). Similarly, in endometrial cancer cells with genetic deletion of MTDH by CRISPR/Cas9 technology, it was observed a marked reduction in FANCD2 protein expression as well as mono-ubiquitin conjugated FANCD2 and FANCI (FIG. 2C). By contrast, MTDH deletion had no effect on mRNA levels on of FANCD2 and FANCI (FIG. 2B), suggesting that the effect of MTDH on FA pathway protein expression is post-transcriptional.

TABLE 2
FANCI and FANCD2 mRNAs sequences pulled-down by PAR-
CLIP(Photoactivatable Ribonucleoside-Enhanced Crosslinking and
Immunoprecipitation) anti-MTDH antibody.

| | | Locus | | Score of RNA-CHIP | |
|---|---|---|---|---|---|
| NT Sequence | Gene | start | End | by MTDH Ab | |
| NM_018193 | FANCI | 68 | 123 | 7.648886902 | CDS(the coding |
| | | 924 | 955 | 5.979785428 | sequences) |
| | | 1023 | 1068 | 7.063917906 | From 91 |
| | | 1349 | 1508 | 8.648887138 | to 3897 |
| | | 1916 | 2098 | 7.063924637 | |
| | | 2206 | 2272 | 7.648886902 | |
| | | 2364 | 2409 | 6.979785428 | |
| | | 3194 | 3258 | 6.063924637 | |
| | | 3370 | 3413 | 7.063917906 | |
| | | 3507 | 3610 | 6.063924401 | |
| | | 4274 | 4356 | 6.063917906 | |
| | | 4360 | 4443 | 6.059907037 | |
| NM_001018115 | FANCD2 | 1353 | 1416 | 9.242527026 | CDS |
| | | 4360 | 4410 | 8.1.62405417 | From 121 |
| | | 4442 | 4499 | 9.162405417 | to 4476 |

Identification of the Region in MTDH that Associates with FANCD2 and FANCI mRNAs

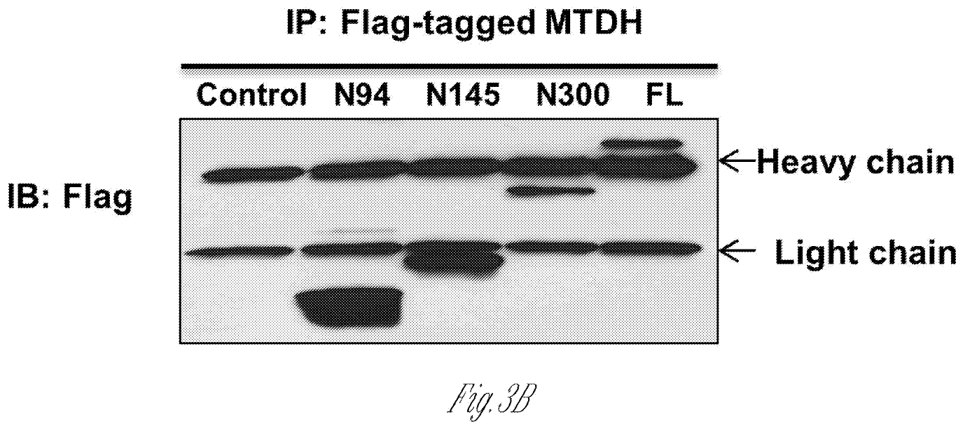

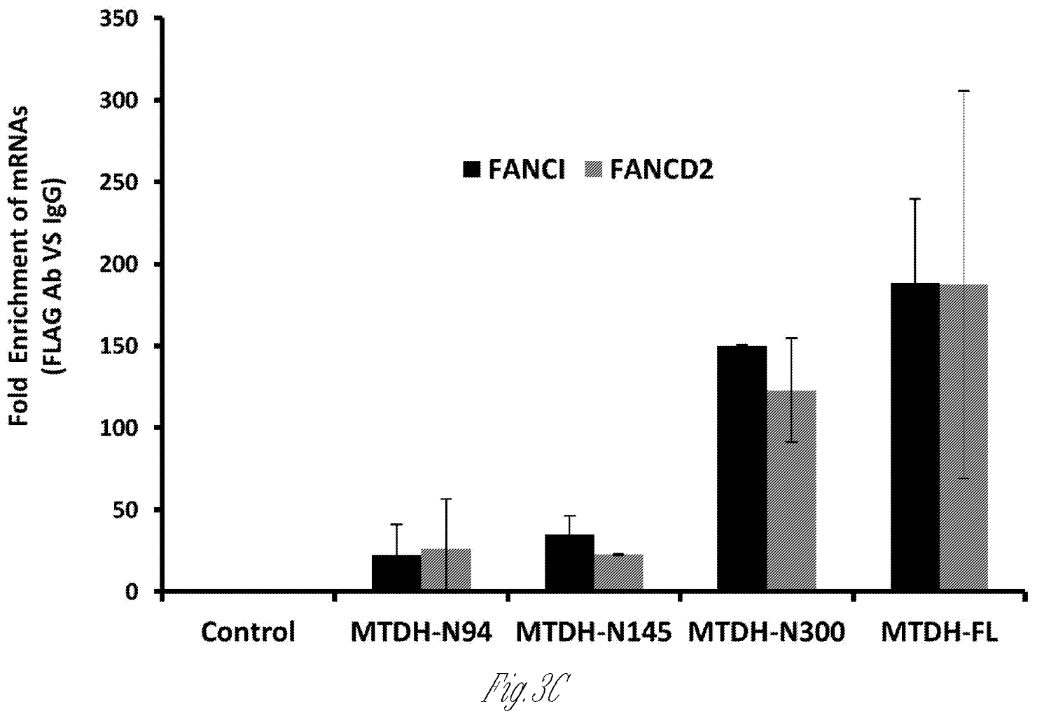

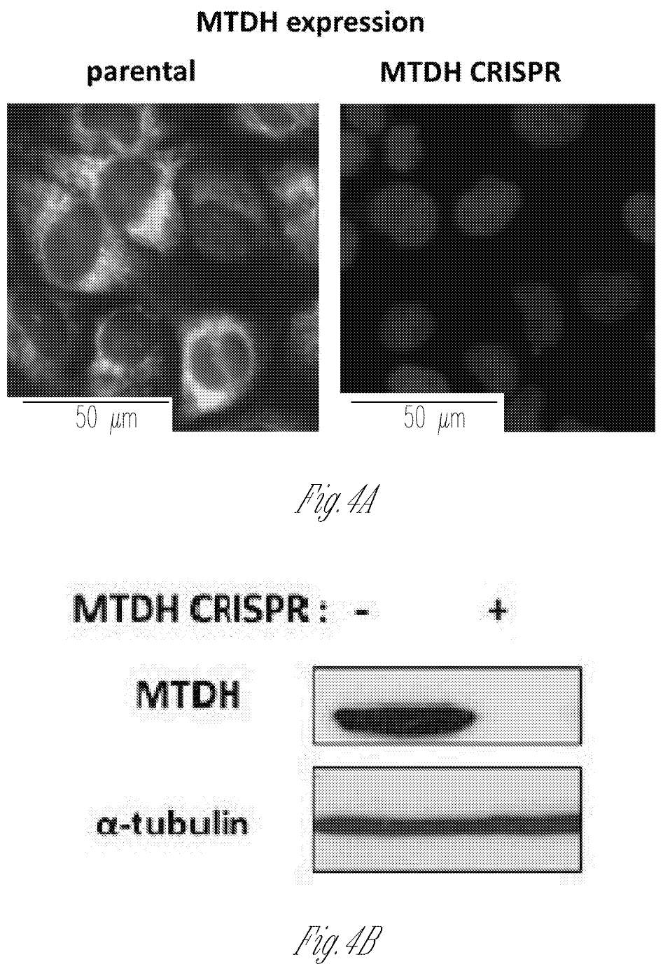

A previous study showed that there are four putative RNA binding regions in MTDH (Meng et al., 2012). To identify the specific region in MTDH that binds mRNAs, FLAG-tagged fragments of MTDH were transiently expressed in Hec50 cells in which endogenous MTDH was knocked down (FIGS. 3 A-B). Protein extracts were subjected to anti-FLAG antibody pull-down followed by RT-qPCR to detect MTDH-bound FANCI and FANCD2 mRNAs. Residues 145-216 were found to be essential for the association of MTDH with FANCD2 and FANCI mRNAs (FIG. 3C).

### MTDH Silencing Increases γ-H2AX Foci Formation and Sensitivity to Cisplatin in Cancer Cells

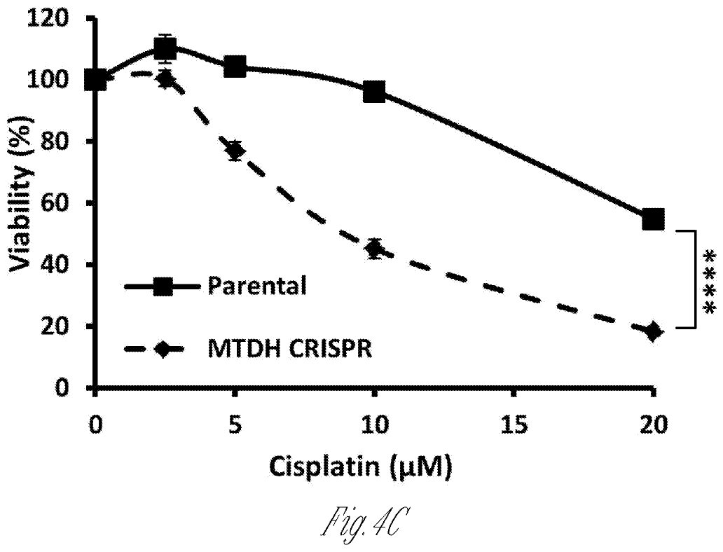

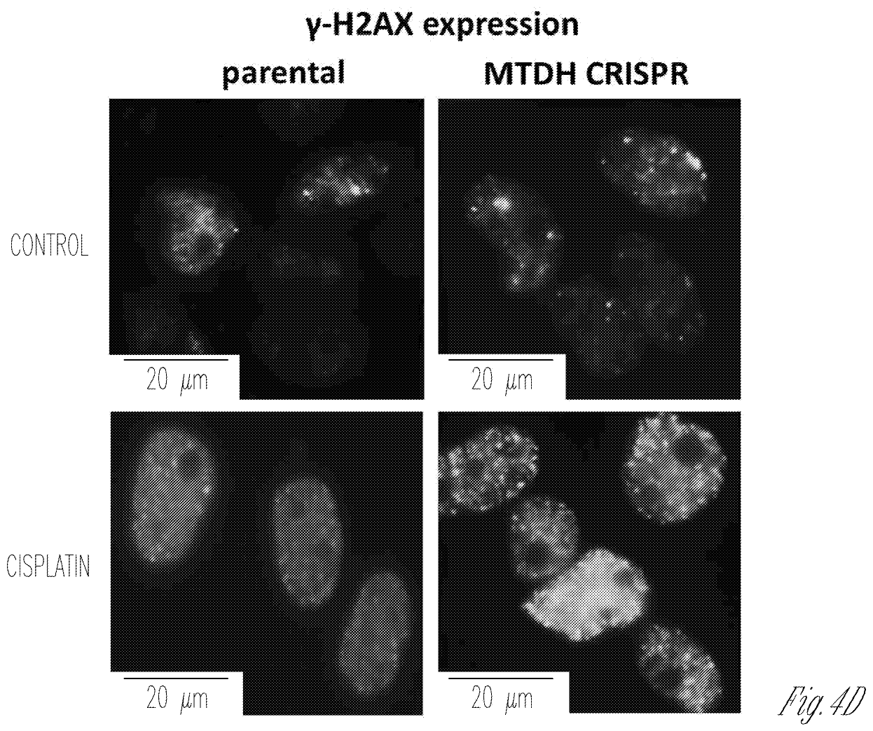

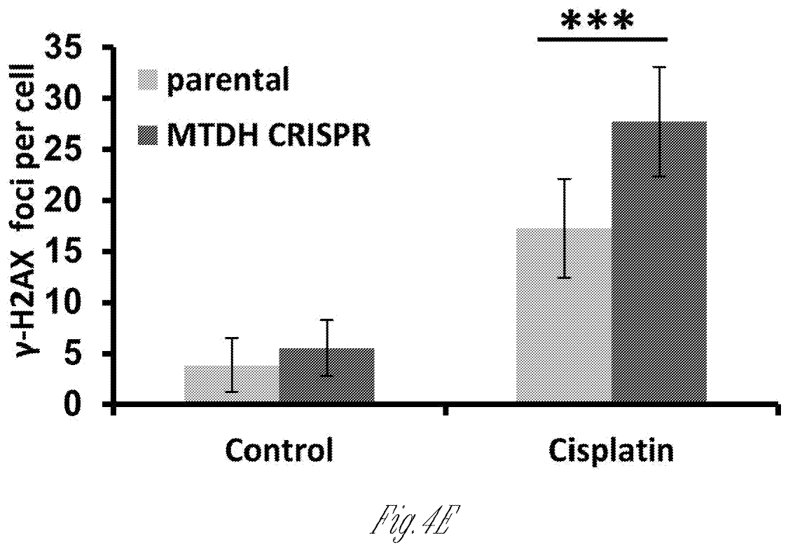

The FA pathway plays a critical role in the repair of DNA cross-link damage induced by chemotherapeutic agents including cisplatin (Kim & D'Andrea, 2012). Consistent with previous reports (Meng et al., 2012), deficiency of MTDH significantly increased sensitivity to the DNA damaging agent cisplatin (FIG. 4A-C). The impact of MTDH on DNA damage repair was directly tested by assessing γ-H2AX foci formation, a standard biomarker to denote an increase in DNA damage (Nowsheen et al., 2009). γ-H2AX foci formation induced by cisplatin was significantly increased in MTDH-deficient Hec50 cells (FIG. 4D, E). From these data, it is concluded that MTDH is required to repair cisplatin induced DNA damage.

### Pristimerin Increases Cisplatin Sensitivity by Downregulating MTDH

Directly targeting MTDH through genetic manipulation is not currently feasible in patients. Therefore, small molecules that can decrease MTDH expression were identified. A recent study in lung cancer cells found that celastrol, a natural agent, promotes proteasomal degradation of FANCD2, thereby increasing sensitivity to DNA crosslinking agents (Moelans et al., 2014). We found that celastrol can also reduce MTDH and FANCI protein levels in cancer cells (FIG. 8). However, celastrol is a leptin sensitizer and leads to weight loss in obese mice (Embdad et al., 2016). To avoid weight loss in cancer patients, we tested another compound with a similar quinonemethide triterpenoid structure, pristimerin.

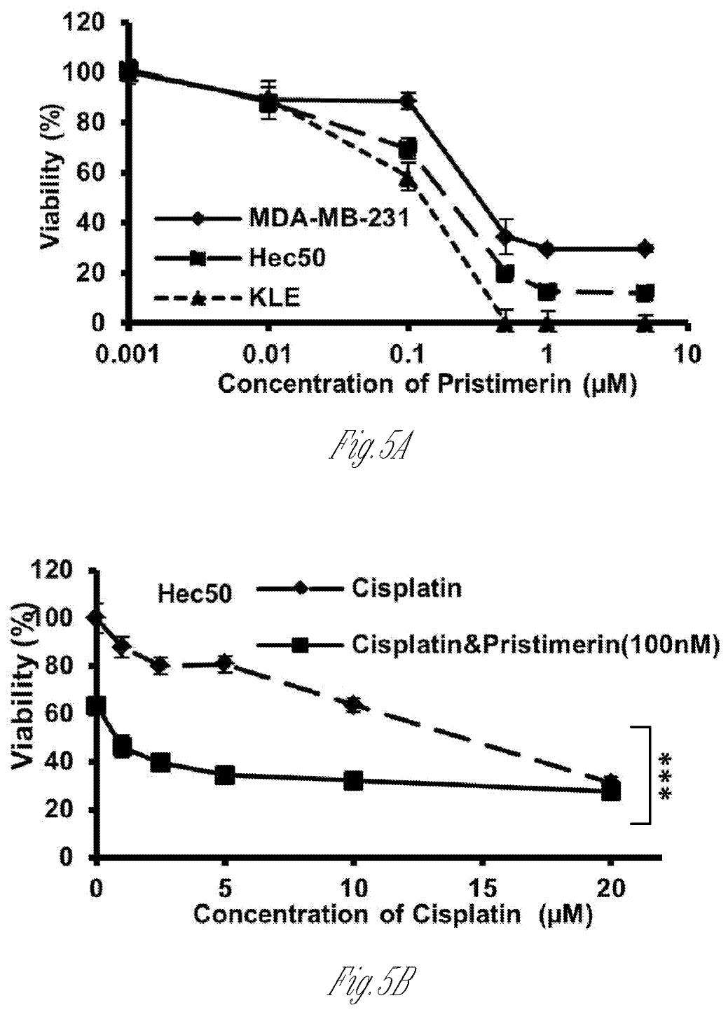

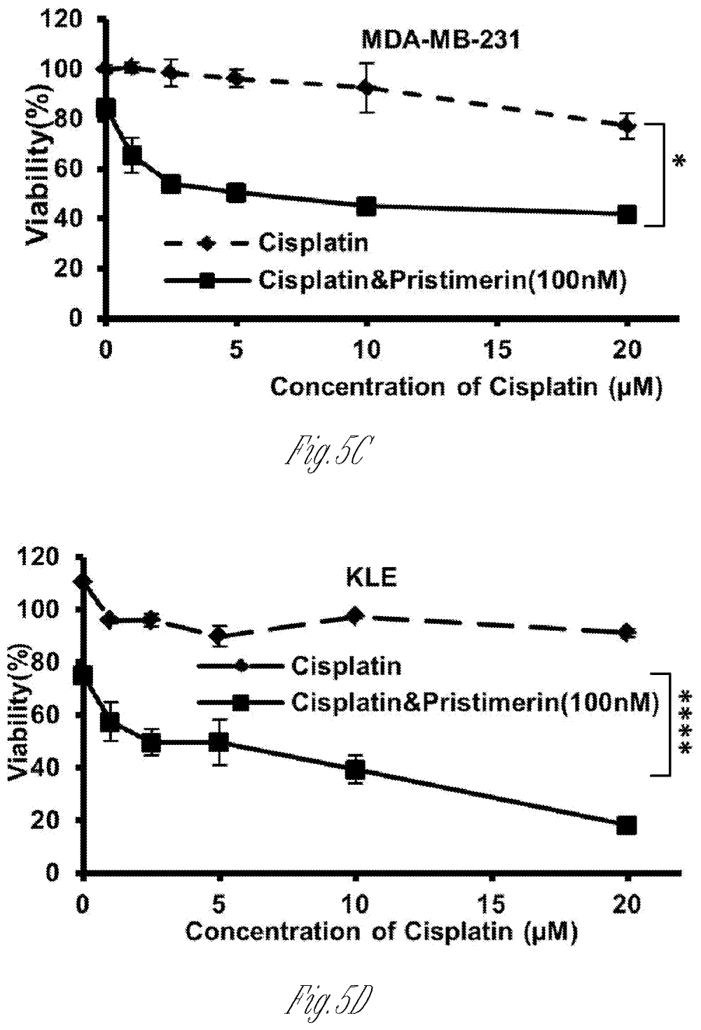

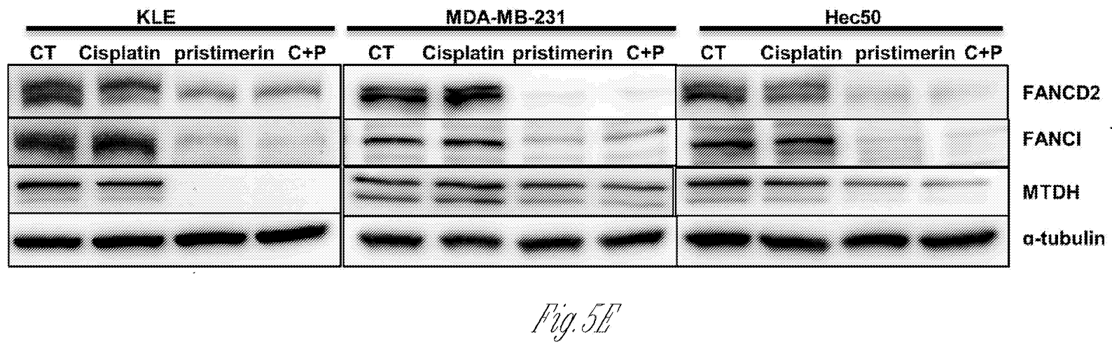

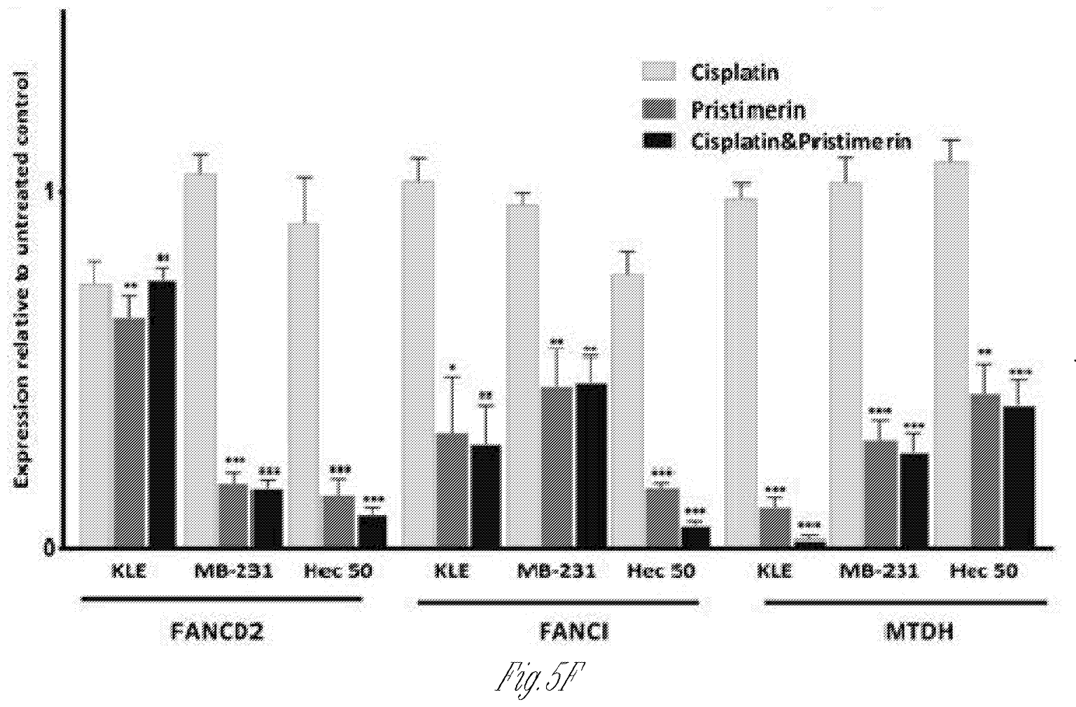

It was established that pristimerin decreases viability of Hec50, MDA-MB-231 and KLE cells, with IC50 values below 1 μM (FIG. 5A). At doses as low as 100 nM, pristimerin increased sensitivity to cisplatin in all three cancer cell lines (FIGS. 5B-D). Importantly, pristimerin decreased MTDH, FANCD2 and FANCI protein levels when used as a single drug or in combination with cisplatin in all three tested cell lines (FIGS. 5E-F). These data demonstrate that treatment with pristimerin is a potential therapeutic approach to overcome the effects of high MTDH expression.

### Quantification and Characterization of Pristimerin-Loaded PLGA Nanoparticles

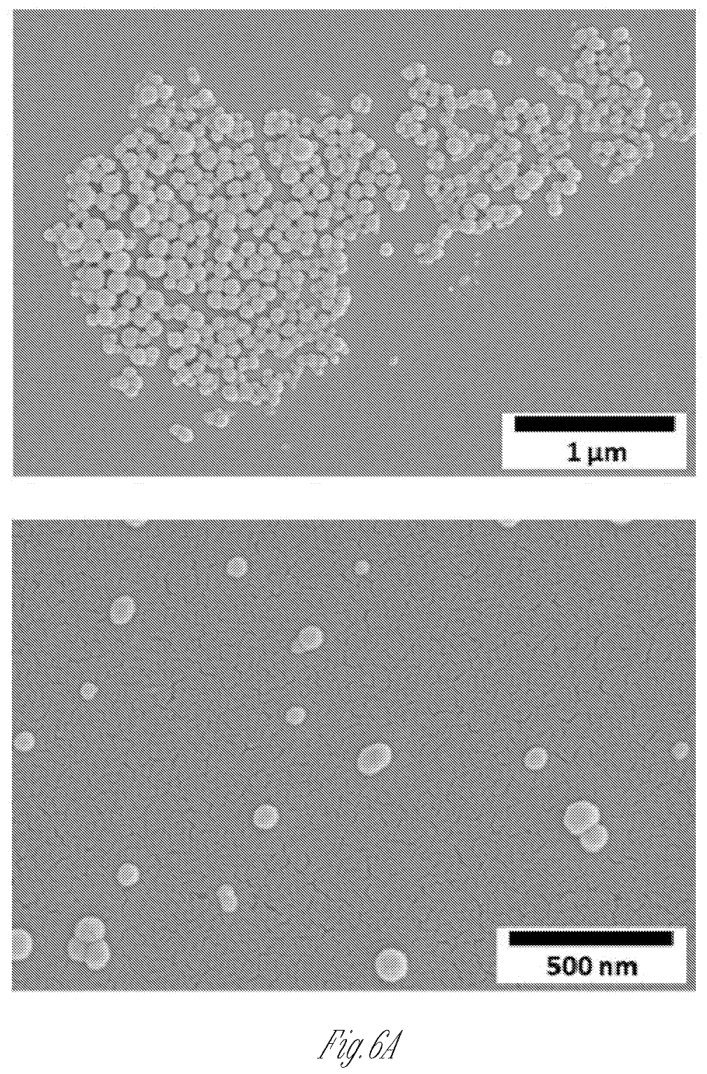

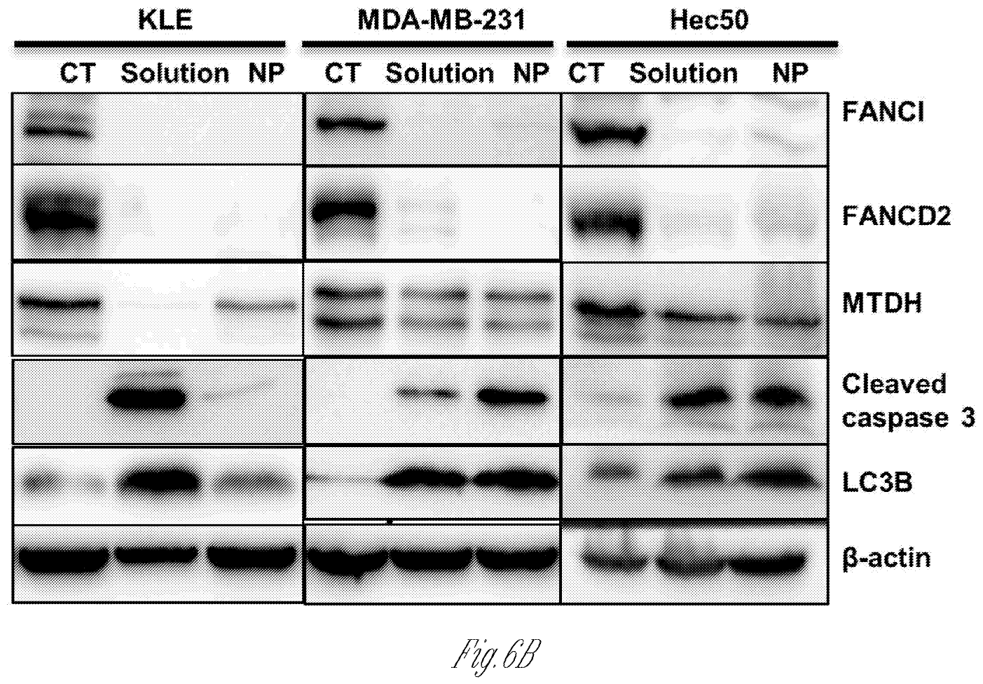

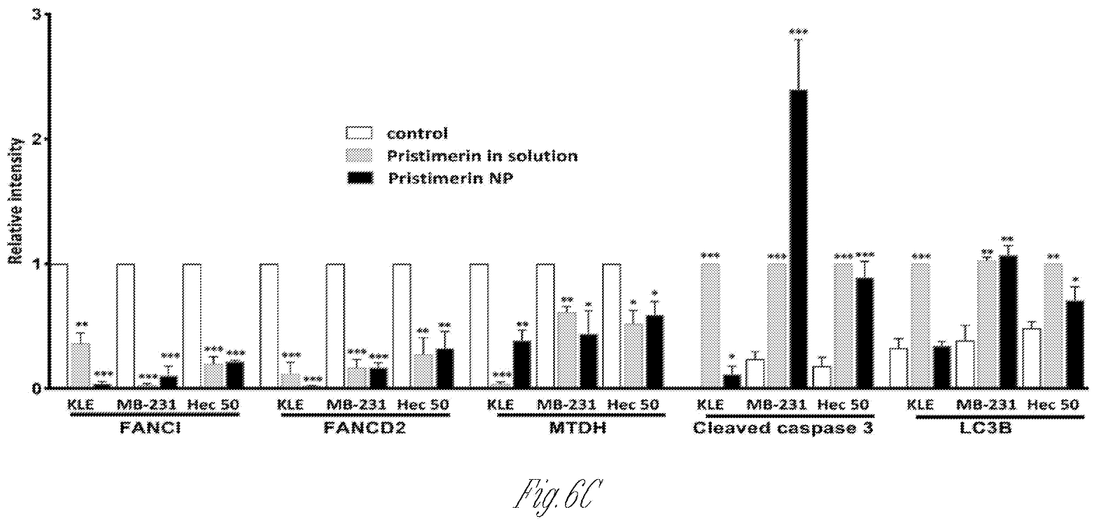

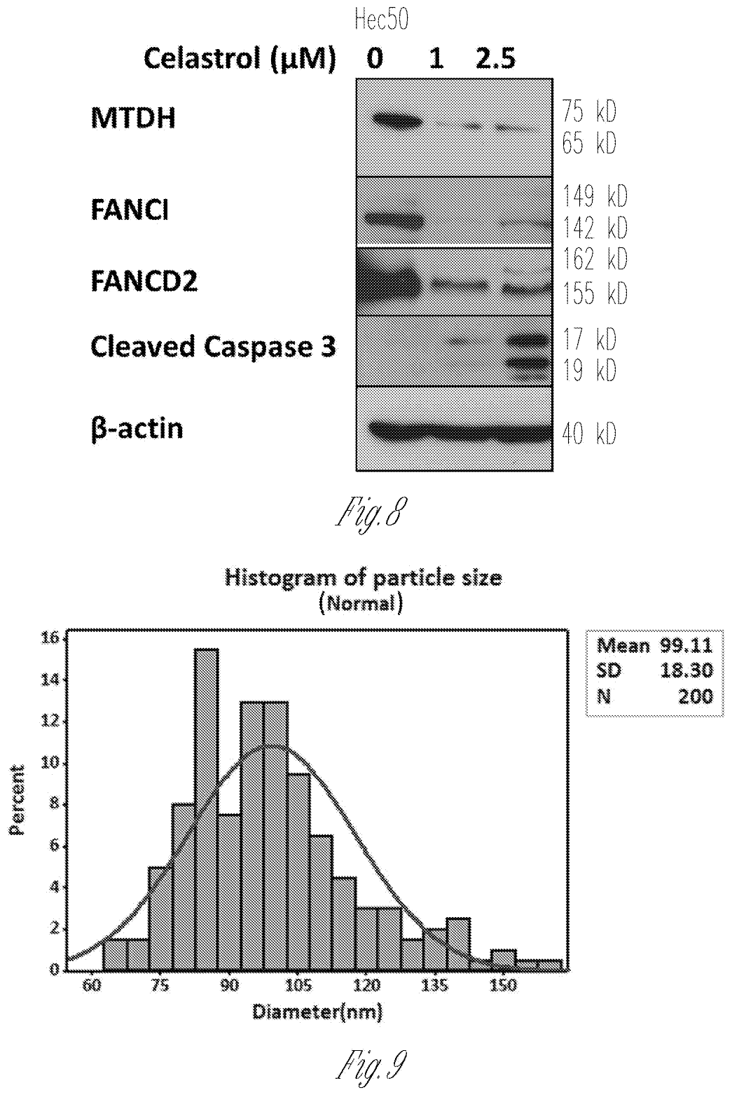

Due to poor solubility and pharmacokinetics, pristimerin in solution did not induce significant tumor growth inhibition in a PDX mouse model of cancer. We therefore used a nanoparticle-based delivery approach to improve the pharmacokinetics and therapeutic efficacy of pristimerin. Pristimerin was loaded into PLGA nanoparticles based on previous studies (Ebeid et al., 2018). The amount of pristimerin-loaded nanoparticles was quantified by HPLC. The drug loading and encapsulation efficiency of pristimerin were 168.70±40.56 μg/mg and 101.22±24.38%, respectively. The particles were characterized by scanning electron microscopy (SEM), which demonstrated that the particle morphology is spherical with a smooth surface (FIG. 6A1). The average particle size was 99.11±18.30 nm (FIG. 6A2). The zeta potential measured by the dynamic light scattering method was −46.82±6.64 mV (FIG. 9).

### Nanoparticle-Delivered Pristimerin Inhibits MTDH, FANCD2 and FANCI in Cancer Cells

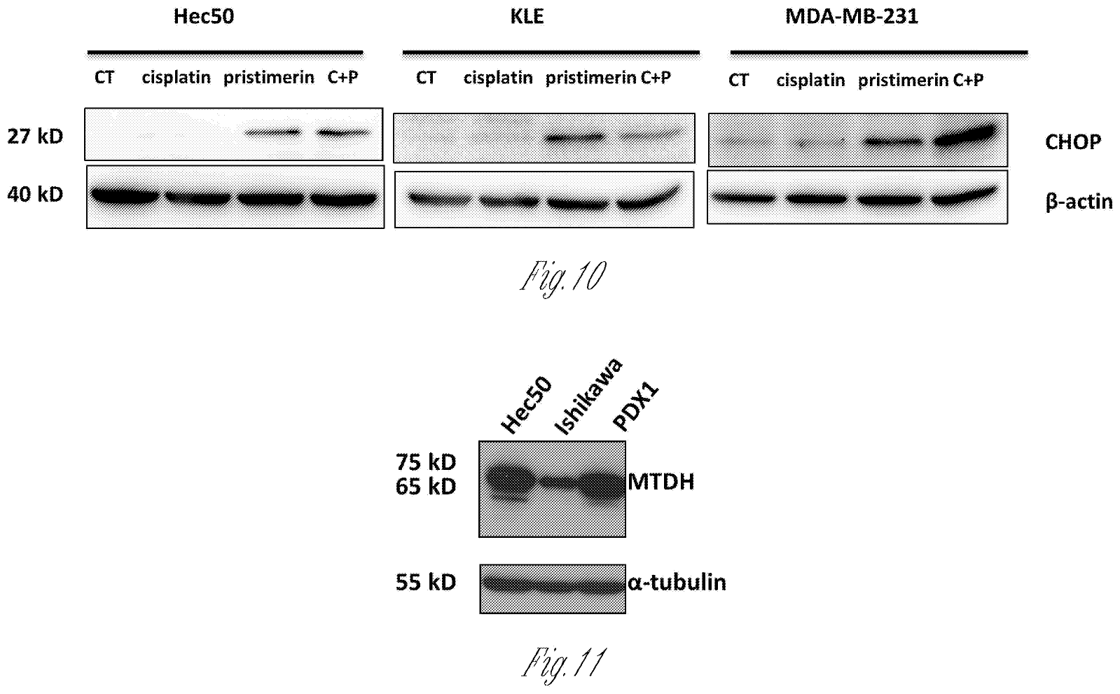

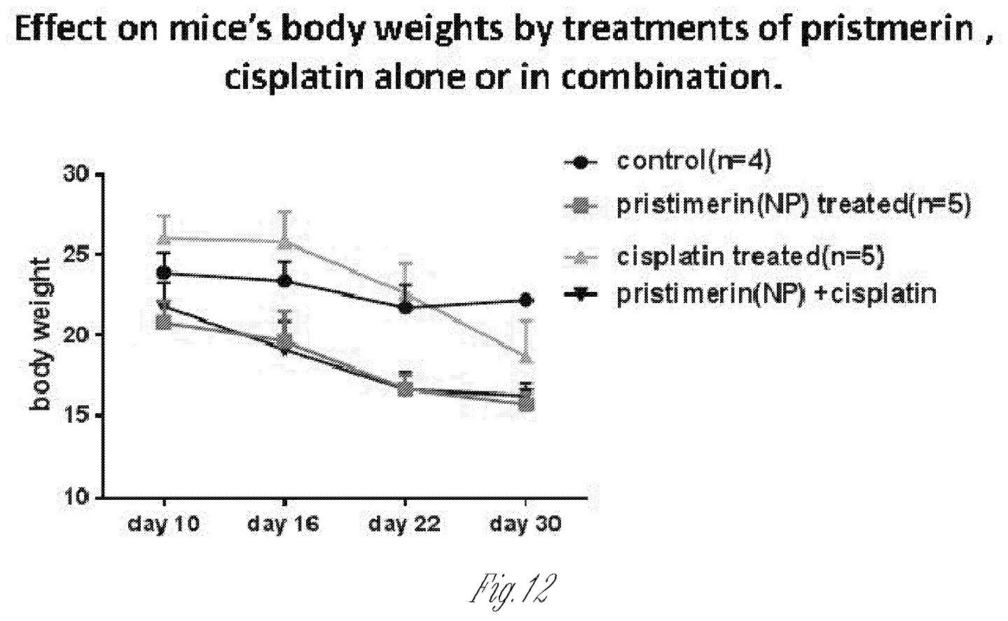

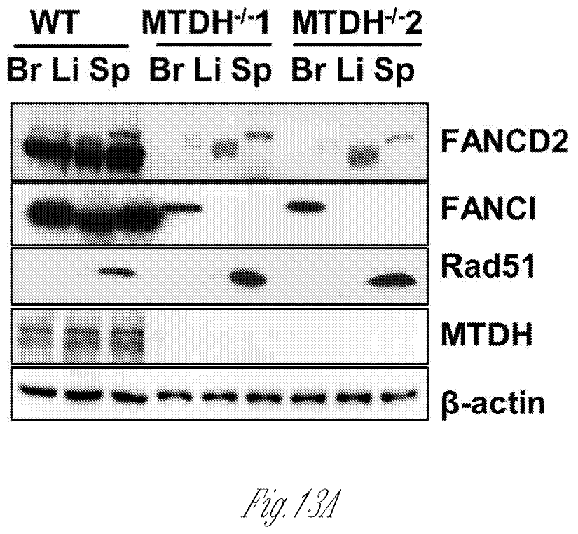

It was first established that pristimerin-loaded nanoparticles reduced protein expression of MTDH, FANCD2 and FANCI to levels similar to those achieved using pristimerin in solution in cell models (FIG. 6B,C). In addition, protein levels of the endoplasmic reticulum (ER) stress biomarker CHOP, the apoptosis biomarker cleaved caspase 3 and the autophagy biomarker LC3B were all increased by treatment of Hec50, MDA-MB-231 and KLE cells with pristimerin-loaded nanoparticles (FIGS. 6B-C and FIG. 10). These data substantiate the efficacy of nanoparticle-delivered pristimerin in downregulating MTDH as well as the involvement of ER stress, apoptosis and autophagy in the mechanism of cell death in response to pristimerin.

Cisplatin Combined with Pristimerin Inhibits Tumor Growth in a Patient-Derived Xenograft Mouse Model

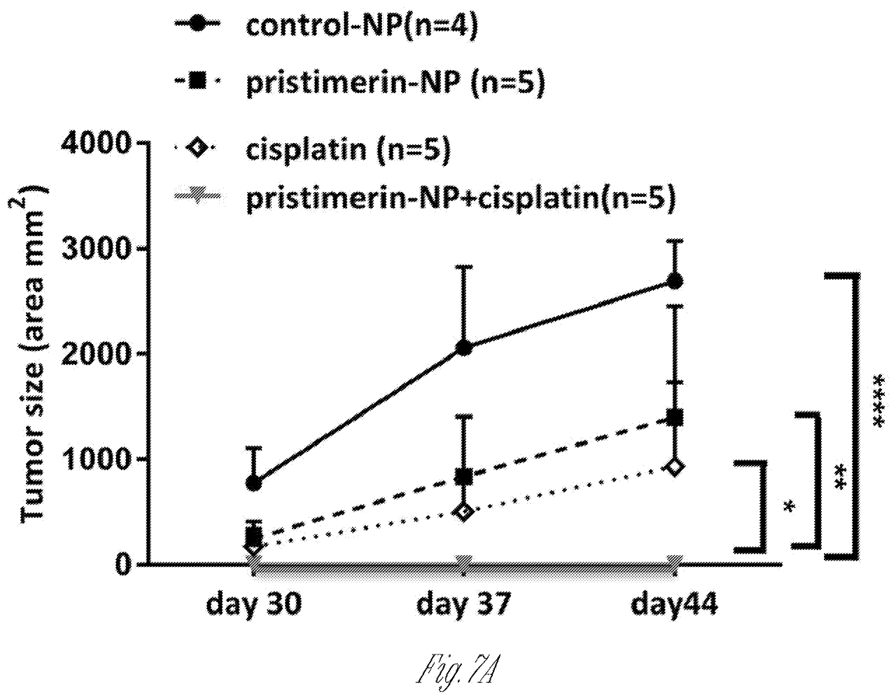

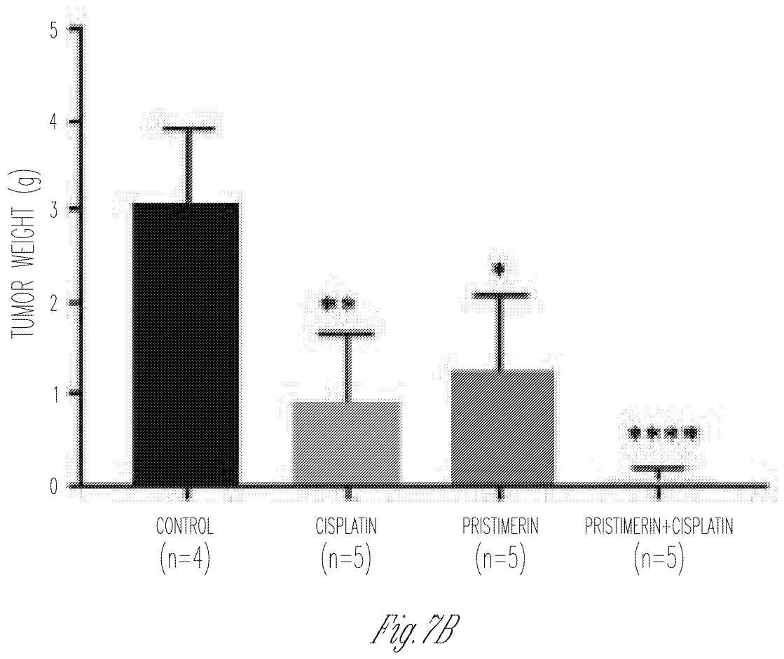

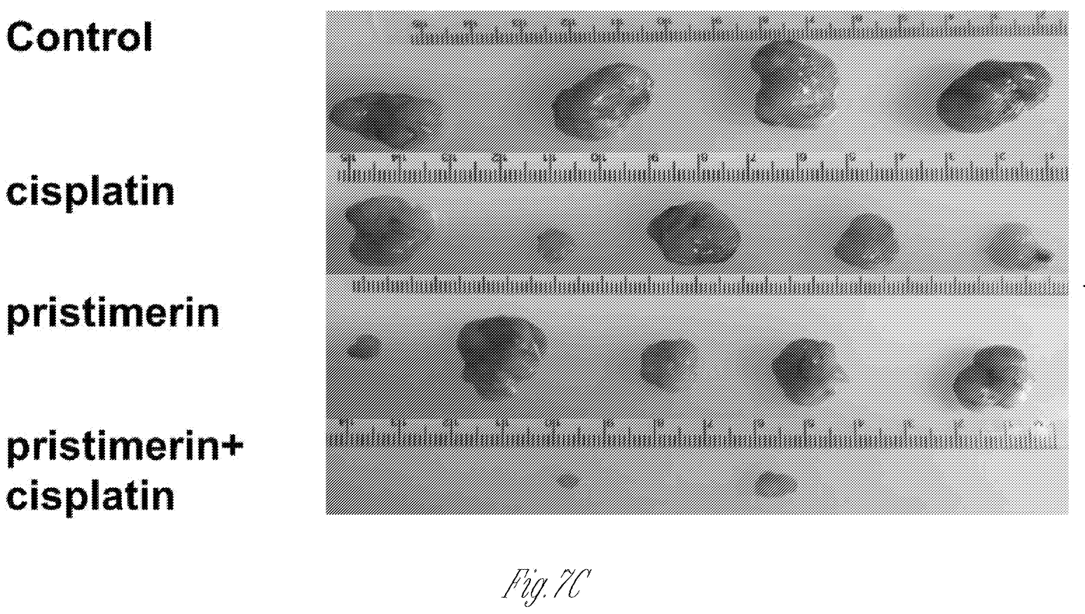

To investigate the effects of pristimerin on tumor growth, studies in a PDX model of serous endometrial cancer were performed. This model, denoted PDX1 herein, was previously developed by implanting a fresh surgically resected endometrial tumor specimen into the subcutis of immunocompromised mice (Luo et al., 2010). PDX1 tumors are subsequently passaged in mice. We first confirmed high expression of MTDH in this model, with levels similar to those observed in Hec50 cells (FIG. 11). Next, immunocompromised mice bearing PDX1 tumors were divided into four different treatment groups: control (empty) PLGA nanoparticles, cisplatin, nanoparticle-loaded pristimerin and the combination of cisplatin with pristimerin-loaded nanoparticles. Treatment with cisplatin or pristimerin alone significantly inhibited tumor growth as compared to control PLGA nanoparticles (p<0.05). However, the combination of cisplatin and nanoparticle-loaded pristimerin further decreased the tumor growth (p<0.001 compared to all other groups, FIG. 7A), with a corresponding reduction in tumor weight at 30 days after treatment (p<0.001) (FIGS. 7B-C). These data identify pristimerin-loaded nanoparticles as a potential treatment to restore sensitivity to cisplatin in tumors with MTDH upregulation.

### Discussion

Platinum compounds are some of the most effective broad-spectrum anti-cancer chemotherapeutic drugs (Desoize & Madoulet, 2002). They function by inducing DNA cross-linking damage in cancer cells in a wide range of cancer types. Unfortunately, drug resistance occurs gradually and frequently in patients whose tumors were initially sensitive to platinum agents (Muggia, 2004; Burger et al., 2011). One mechanism of resistance is an increased ability of cancer cells to repair platinum-induced DNA damage (Tortorell et al., 2018; Hellweg et al., 2019). DNA interstrand-crosslink damage is mainly recognized by proteins in the FA pathway and subsequently repaired by the homologous recombination repair (HRR) pathway (Ceccaldi et al., 2016; Deans & West, 2011; Venkitaraman, 2004; Thompson, 2005; Taniguchi et al., 2002). The majority of studies of FA-mediated DNA repair in cancer focus on inactivating mutations in FA genes. Indeed, the 17 FA genes in the FA pathway are frequently mutated across 68 DNA sequence datasets of non-Fanconi Anemia human cancers, at a rate in the range of 15 to 35% (Shen et al., 2015). BRCA2 is among these 17 genes, and studies in ovarian cancer demonstrate that tumors with mutations in BRCA2 are initially sensitive to platinum compounds due to loss of DNA repair capabilities (Sakai et al., 2008; Sakai et al., 2009).

Herein it is reported that this canonical DNA repair mechanism can also be co-opted to drive chemoresistance. Specifically, it was found that overexpression of MTDH up regulates FANCD2 and FANCI by interacting with and promoting translation of FANCD2 and FANCI mRNAs. By upregulating these DNA repair proteins, MTDH accomplishes massive resistance to DNA-damaging agents by endowing cancer cells with an enhanced ability to repair damaged DNA. Consistent with the present findings, others have found that FANCD2 expression is up regulated and correlates with poor outcome in hepatocellular carcinoma (Komatsu et al., 2017). Despite a loss of protein expression, changes in mRNA levels of FANCD2 or FANCI in MTDH-deficient cells were not detected. Hence, it was concluded that MTDH regulates FA family proteins at the post-transcriptional level. Consistent with this interpretation, MTDH has been found to bind to many sequences in the coding region and 3-terminal untranslated region of FANCI (Table 2 herein (see Hsu et al., 2018)).

Since MTDH regulates the expression of a cadre of FA pathway factors through its RNA binding properties, MTDH may make for a good therapeutic target by which to increase sensitivity to platinum compounds. Currently, no MTDH specific inhibitors are available given the lack of canonical catalytic domains in MTDH. The discovery that pristimerin can efficiently reduce expression of MTDH and FA pathway proteins provides a potential solution to repurpose this anti-inflammatory drug to combine with chemotherapy. Pristimerin is a natural triterpenoid isolated from the Celastraceae and Hippocrateaceae plant families and is widely used in traditional Chinese medicine as an anti-inflammatory medication (Shkreta & Chabot, 2015). Multiple preclinical studies in a wide range of cancer types, including breast cancer, colon cancer, prostate cancer and pancreatic cancer, confirm the anti-tumor activity of pristimerin (Park et al., 2018). Mechanistic studies have suggested that the anti-inflammatory activity of pristimerin is accomplished through inhibition of the well-known pro-inflammatory transcription factor NF-κB via inhibition of the NF-κB inhibitor IKK (Hui et al., 2014). In addition, pristimerin has been shown to inhibit chymotrypsin-like protease activity (Tiedeimann et al., 2009), suggesting that pristimerin is a dual proteasome and NF-κB inhibitor. Of note, NF-κB regulates expression of MTDH by binding to the promoter of the MTDH gene (Sarkar et al., 2008). Therefore, pristimerin may accomplish the reduction of MTDH expression by interfering with NF-κB-mediated transcription of this gene.

To enhance drug solubility, stability and accumulation in the tumor, a nanoparticle formulation was used to deliver pristimerin to tumors in vivo (Ebeid et al., 2018; Cheng et al., 2007). Nanoparticles have been utilized for delivering therapeutic and diagnostic agents. Nanoparticles may offer a superior dissolution profile of their payload due to their unique size range that governs a vast increase in the exposed surface area to the dissolution medium. Nanoparticles prepared from natural or synthetic polymers modify drug release and create a sustained or controlled release profile. The specific nanoparticle formulation used to deliver pristimerin herein, which comprises PLGA at a monomer ratio of 75:25 and tocopheryl polyethylene glycol succinate (TPGS) surfactant, improves therapeutic efficacy of pristimerin through enhanced drug uptake and accumulation. Other surfactants may be employed. In one embodiment, the surfactant includes but is not limited to C8/C10 glycerol and PEG esters, e.g., Cremophor, Solutol HS15, labrosol, Softigen 767 or aconnon E, sucrose esters, e.g., sucrose monolaurate or sucrose monooleate, or polysorbates, e.g. Tween 80 or Tween 20. In one embodiment, the surfactant includes but is not limited to d-α-tocopherol poly-(ethylene glycol) succinate (Vit-E-PEG), poly(ethylene oxide) 20 sorbitan monooleate (Tween 80), cetyltrimethylammonium bromide (CTAB), poly(ethylene oxide) 35 modified castor oil (Cremophor EL), polyethylene glycol 15-hydroxystearate (Solutol HS 15), poly(ethylene glycol) hexadecyl ether (Brij 58), sodium 1,4-bis (2-ethylhexoxy)-1,4-dioxobutane-2-sulfonate (AOT), Tween 80 (p<0.0001), CTAB (p=0.004), poly(ethylene oxide) 20 sorbitan monolaurate (Tween 20), Cremophor EL or sodium carboxymethyl cellulose (NaCMC). In one embodiment, a carrier that comprises a surfactant or a polymer may be employed to deliver the therapeutic, including but not limited to alkyl (C12-16) dimethylbenzylammonium chloride (Hyamine), cetyltrimethylammonium bromide (CTAB), D-α-tocopherol poly-(ethylene glycol) succinate (Vit-E-PEG), magnesium stearate, poly-(ethylene glycol) hexadecyl ether (Brij 58), poly(ethylene oxide) 20 sorbitan monolaurate (Tween 20), poly(ethylene oxide) 20 sorbitan monooleate (Tween 80), poly(ethylene oxide) 35 modified castor oil (Cremophor EL), polyethylene glycol 15-hydroxystearate (Solutol HS 15), sodium 1,4-bis (2-ethylhexoxy)-1,4-dioxobutane-2-sulfonate (AOT), sodium caprylate (NaCap), sodium deoxycholate (NaDC), sucrose palmitate (Sisterna 16), sucrose stearate (Sisterna 11), hydrolyzed gelatin (HG, MW=1980 g/mol), hydroxypropyl cellulose (HPC, MW=80 000 g/mol), hydroxypropylmethyl cellulose (HPMC, MW=10 000 g/mol), poly(ethylene oxide) 101-block-poly(propylene oxide) 56-block-poly(ethylene oxide) 101 (Pluronic F127, MW=12 600 g/mol), poly(ethylene oxide) 80-block-poly(propylene oxide) 27-block-poly(ethylene oxide) 80 (Pluronic F68, MW=8400 g/mol), poly(vinyl alcohol) (80% hydrolyzed PVA, MW=9500 g/mol), poly(vinyl alcohol)-graft-poly(ethylene glycol) copolymer (Kollicoat, MW=45 000 g/mol), poly-(vinylpyrrolidone) (PVP K30, MW=40 000 g/mol), poly-(vinylpolypyrrolidone) (PVPP), poly(ethylene glycol) (PEG, MW=1000 g/mol), or sodium carboxymethylcellulose (NaCMC, MW=90 000 g/mol). Regarding cancer treatment, nanoparticles less than 200 nm in diameter may offer superior accumulation at the tumor site due to the enhanced permeability and retention (EPR) effect (Acharya & Sahoo, 2011).

In conclusion, the present data support the role of MTDH overexpression as a mechanism that leads to resistance to chemotherapy via its RNA binding function. It is also demonstrated that inhibition of MTDH expression leads to a significant reduction in FA DNA repair proteins, and this effect can be phenocopied by treating with pristimerin loaded nanoparticles. Thus, the nanoparticles may be employed in a therapeutic strategy to improve chemosensitivity.

### Example 2

### Material and Methods

Analysis of MTDH, FANCD2 and FANCI in TCGA data

UCSC Xena browser (https://xena/ucsc.edu) is a public hub with detail online instruction that was used to analyze the correlation of MTDH amplification with MTDH, FANCD2 and FANCI expression in endometrial cancer and breast cancer patients in TCGA (The Cancer Genome Atlas) (Goldman et al., 2018). Gene expression was determined by comparing transcript-level expression of MTDH, FANCD2 and FANCI based on the RNA-sequencing data in the TCGA dataset. MTDH amplification was determined by analyzing copy number variation (CNV) for MTDH after removal of germane CNVs.

### Cell Line and Culture Conditions

Hec50 uterine serous carcinoma cells were kindly provided by Dr. Erlio Gurpide in 1991 (New York University). KLE uterine serous carcinoma cells and MDA-MB-231 breast cancer cells were purchased from American Type Culture Collection in 2009 (ATCC, Manassas, Va.). Hec50 and MDA-MB-231 cells were cultured in Dulbecco's modified Eagle's medium (DMEM) supplemented with 10% fetal bovine serum (FBS, Gibco, Grand Island, N.Y.) and penicillin/streptomycin. KLE cells were cultured in RPMI-1640 (Gibco) supplemented with 10% FBS and penicillin/streptomycin. Cell line authentication is performed yearly for all studied lines using the CODIS marker testing (BioSynthesis, Lewisville, Tex.). Mycoplasma testing is also performed annually by the University of Iowa DNA Sequencing Core facility. Cells were used no more than 10 passages from thawing to the completion of all experiments.

### Western Blotting

Cells were scraped into ice-cold RIPA buffer (50 mM Tris-HCl pH 7.4, 150 mM NaCl, 2 mM EDTA, 1% NP-40, 0.1% SDS with protease inhibitor) and sonicated three times. Lysates were then centrifuged at 12,000×g for 15 minutes at 4° C. and protein was quantified by BCA assay (Thermo Fisher Scientific, Waltham, Mass., USA). Samples were separated on 10% or 7.5% SDS-PAGE gels then transferred to a nitrocellulose blotting membrane, which was blocked with 5% nonfat milk and incubated overnight at 4° C. with primary antibodies. Anti-FANCD2 (1:2000, #NB 200-182) and anti-FANCI (1:500, #ab74332) were obtained from Novus Biologicals (Centennial, Colo., USA). Anti-RAD51 (1:1000, #8875), anti-LC3B (1:1000, #3868) and anti-cleaved caspase 3 (1:1000, #9661) were from Cell Signaling Technology (Danvers, Mass.). Anti-b-actin (1:10,000, #A5441) was from Sigma (St. Louis, Mo.). Anti-MTDH (1:250, #517220) was from Santa Cruz Biotechnology (Dallas, Tex.). Membranes were further incubated with appropriate secondary antibodies at 1:10,000 (#7076 and #7074, Cell Signaling Technology) at room temperature for 2 hours. Protein bands were detected using the Bio-Rad ChemiDoc system, and densitometry was analyzed with BioRad Image Lab Software (Bio-Rad Laboratories, Hercules, Calif.). For the mice tissue, tissues were dissected on ice, grinded with a mortar and pestle in liquid nitrogen and transferred 20 mg tissue powder to 1.5 ml Eppendorf tube in 1 ml of ice-cold RIPA buffer and homogenize using electric homogenizer. Lysates were then centrifuged at 12,000×g for 15 minutes at 4° C. The supernatant was collected in fresh tube on ice. Protein samples were analyzed with SDS-PAGE gels as described above.

### Cell Viability Assays

Cell viability was determined by WST-1 assay. Cells were seeded into 96-well plates (1×10⁴ cells per well) then treated with cisplatin (Fresenius Kabi Oncology Ltd., Haryana, India) or the combination of cisplatin with pristimerin in solution (Cayman Chemical, Ann Arbor, Mich.). Cell viability was evaluated using the cell proliferation reagent WST-1 (Roche, Germany) according to the manufacturer's protocol. Absorbance was measured with a microplate reader (BioRad). Data were calculated as percent (%) viability relative to untreated control, which was set at 100%.

### XTT Assay

Cells expressing scrambled sgRNA and multiple MTDH knockout clones were seeded into 96-well plates (1×10³ cells per well). Cell growth was monitored by measuring daily over 5 days by XTTassay. XTT (GoldBio, St Louis, Mo.) solutions were made fresh each day by dissolving XTT in cell culture medium at 1 mg/ml. PMS (Sigma) was made up as a 100 mM solution in phosphate buffered saline and used at a final concentration of 25 μM. PMS was added to the XTT solution immediately before use and cells were incubated for 1-2 hours at 37° C. The reaction was placed on a shaker for a short period of time to mix the dye in the solution. Absorbance was measured at 450 nm immediately.

### Immunofluorescence (IF) Staining

Hec50 cells were seeded on coverslips then fixed with 2% paraformaldehyde for 20 minutes. Coverslips were rinsed 3 times with 1 ml PBS and incubated with 80% ice-cold methanol for 1 hour, followed by permeabilization for 25-30 minutes with 0.2% Triton X-100. Cells were blocked with 3% BSA then incubated with specific antibodies at 4° C. overnight. Anti-MTDH (1:100, #14065), antiphospho-histone H2AX (Ser139) (1:400, #9718) were from Cell Signaling Technology (Danvers, Mass.). Then, cells were incubated with Alexa Fluor 546-conjugated anti-rabbit secondary antibody (1:200, Cell Signaling Technology) at room temperature for 2 hours; nuclei were stained using mounting solution with DAPI (Vector Laboratories). Visualization was performed on a Zeiss 710 confocal microscope.

### Animal Studies

All animal studies were performed under animal protocols #7051085 approved by the University of Iowa Institutional Animal Care and Use Committee (Iowa City, Iowa). MTDH knockout mice were generated as described previously (Meng et al., 2015). Male mice at 20 weeks of age were euthanized and the spleen, brain and liver were removed and immediately snap frozen in liquid nitrogen. Tissue was ground to a fine powder in liquid nitrogen, sonicated in ice-cold RIPA buffer and then protein was extracted for Western blotting. A patient-derived xenograft (PDX) model of endometrial cancer (PDX1) has been previously described (Luo et al., 2010). NOD.Cg-Prkdcscid Il2rgtm1Wjl/SzJ (NSG, Jackson Laboratories, Bar Harbor, Me.) immunodeficient female mice at 8 weeks of age were injected with passage 3 of PDX1 tumor tissue (10 mg/100 ml medium) into the right flank subcutaneously. The mice were randomly divided into 4 groups, with 5 mice per drug treated group, and 4 mice comprised the empty nanoparticle control group. Treatment was started on day 15 after engraftment of cells. The dose of pristimerin delivered in nanoparticles was 3 mg/kg for each mouse and was administered by intravenous (IV) injection twice a week for a total of 4 weeks. The dose of cisplatin was 2.5 mg/kg for each mouse and was administered by intraperitoneal (IP) injection twice a week for a total of 4 weeks. After 2 weeks of treatment, tumors were measured weekly using calipers, and volumes were calculated using the formula length×width/2.

### MTDH Silencing by CRISPR Editing

MTDH expression knockout using CRISPR/Cas9 was achieved as described previously (Kavlashvili et al., 2016). The sgRNA CAAAACAGTTCACGCCATGA (SEQ ID NO:1) targeted the coding region of the MTDH gene at 97686713 to 97686733 (Sequence ID: NC_000008.11 at Homo sapiens chromosome 8, GRCh38.p12). The sgRNA was cloned into lentiCRISPRv1 (Addgene Plasmid 49535, Addgene, Watertown, Mass., USA). The viral vectors were produced in HEK293T cells following the manufacturer's protocol. Endometrial cancer cells of Hec50 were infected with the lentivirus and cultured in the presence of puromycin. Single cell clones were selected by limiting dilution. MTDH deletion was confirmed by qPCR and by Western blotting.

### Preparation of Pristimerin-Loaded Nanoparticles

Pristimerin-loaded Poly (DL-lactide-co-glycolide) (PLGA) nanoparticles were prepared using the nanoprecipitation method as described previously (Ebeid et al., 2018). Briefly, 2 mg of pristimerin and 20 mg of 75:25 Poly (DL-lactide-co-glycolide) (Lactel Absorbable Polymers, Birmingham, Ala.) were dissolved in 3.4 ml of acetone, sonicated for 10 minutes (Branson® 5200), and then mixed with 0.6 ml of 97% ethanol. This organic solution was added drop wise into a stirred aqueous solution prepared by mixing 20 ml distilled water with 0.6 ml of 1% (w/v) D-α-Tocopherol polyethylene glycol 1000 succinate (Sigma Aldrich). The organic solvent in the nanoparticle suspension was evaporated under reduced pressure of 50 mBar for 6 hours using a rotary evaporator (Heidolph, Laborota 4000-efficient). Nanoparticles were then washed 4 times using Amicon ultra-15 centrifugal filter units (MW cutoff ¼ 100 kDa (EMD Millipore)) by centrifugation at 500 g for 20 min (Eppendorf® centrifuge 5804 R). Pristimerin-loaded nanoparticles were freshly prepared before each experiment.

### Quantification of Pristimerin Loading

In order to determine pristimerin loading per mg of nanoparticles, freshly prepared pristimerin-loaded nanoparticles were frozen overnight and then lyophilized using a Labconco freeze dryer (FreeZone 4.5). Known amounts of lyophilized pristimerin-loaded nanoparticles were dissolved in acetonitrile, and then pristimerin loading was quantified using high performance liquid chromatography (HLPC, Waters, 2690 separations module) equipped with an ultraviolet detector (Waters, 2487 Dual λ absorbance detector) using 425 nm as the detection wavelength. The column was a Symmetry Shield™ RP 18, 5 μm, 4.6×150 mm. Isocratic elution was carried out using a mobile phase consisting of a mixture of methanol and ultrapure water+0.1% (v/v) phosphoric acid (80:20) at a flowrate of 1 ml/min with 10 μl as the injection volume. A standard curve of known concentrations of pristimerin solution in acetonitrile was generated and used to determine pristimerin loading in the nanoparticles.

Drug loading and encapsulation efficiency (% EE) were calculated from Eqs. (1) and (2), respectively. In the equations, nanoparticles are abbreviated as “NPs.”

$$\begin{matrix}Drug loading ( \frac{μg of drug}{mg of NPs} ) = \frac{Amount of pristimerin in NPs ( μg )}{Total weight of NPs ( mg )} & ( 1 ) \\ Encapsulation efficiency ( \% ) = \frac{Amount of pristimerin in NPs ( mg )}{Initial amount of pristimerin ( mg ) } \times 100 & ( 2 )\end{matrix}$$

### Pulldown of MTDH-Associated RNAs by Flag Tagged MTDH-Fragments

MTDH fragments were established by cloning PCR products in pCMV6 vector (Origene, Rockville, Md.) and transfected to Hec50 cells with MTDH short hairpin RNA knockdown (Meng et al., 2012). Magna RIPTM (RNA-binding protein immunoprecipitation) kit (Millipore, Bedford, Mass.) and real time PCR were used to pull down MTDH-associated RNAs and to identify mRNAs that associate with MTDH per the manufacturer's protocol described in Meng et al. (2012). Antibody against MTDH (40-6500, 5 μg/l ml, ThermoFisher, Inc., Waltham, Mass.) or FLAG antibody M2 (Millipore Sigma, St. Louis, Mo.) was used to pull down MTDH-associated mRNAs, and anti-IgG (5 μg/1 ml, Millipore, Bedford, Mass.) was used as a negative control. FANCD2 and FANCI were detected by RT-qPCR. 18srRNA was used as control. Primer details are provided in Table 3.

TABLE 3
qPCR primers to quantity mRNA expression
of MTDH, FANCD2, FANCI.

| | | Primer length(bp) | |
|---|---|---|---|
| | | PCR product | |
| Primer | Sequence | length(bp) | |
| FANCD2 | GTTCGCCAGTT | 21 | 175 |
| Forward | GGTGATGGAT | | |
| | (SEQ ID NO: 2) | | |
| FANCD2 | GGGAAGCCTGT | 20 | |
| Reverse | AACCGTGAT | | |
| | (SEQ ID NO: 3) | | |
| MTDH | AAGCAGTGCAA | 22 | 111 |
| Forward | AACAGTTCACG | | |
| | (SEQ ID NO: 4) | | |
| MTDH | GCACCTTATCA | 21 | |
| Reverse | CGTTTACGCT | | |
| | (SEQ ID NO: 5) | | |
| FANCI | CACCACACTTA | 20 | 60 |
| Forward | CAGCCCTTG | | |
| | (SEQ ID NO: 6) | | |
| FANCI | ATTCCTCCGGA | 19 | |
| Reverse | GCTCTGAC | | |
| | (SEQ ID NO: 7) | | |

### Statistical Analysis

Kaplan Meier analysis was used to determine the association of MTDH amplification with survival in endometrial cancer TCGA dataset. Two-sided paired t-tests were used to compare test sets with controls. Two-way ANOVA was used for comparisons between control and treatment over a range of doses or times. P values are denoted as follows: “*”<0.05, “*”<0.01, “***”<0.001, “****”<0.0001.

### Results

### MTDH Depletion Causes a Reduction in FANCD2 and FANCI Proteins

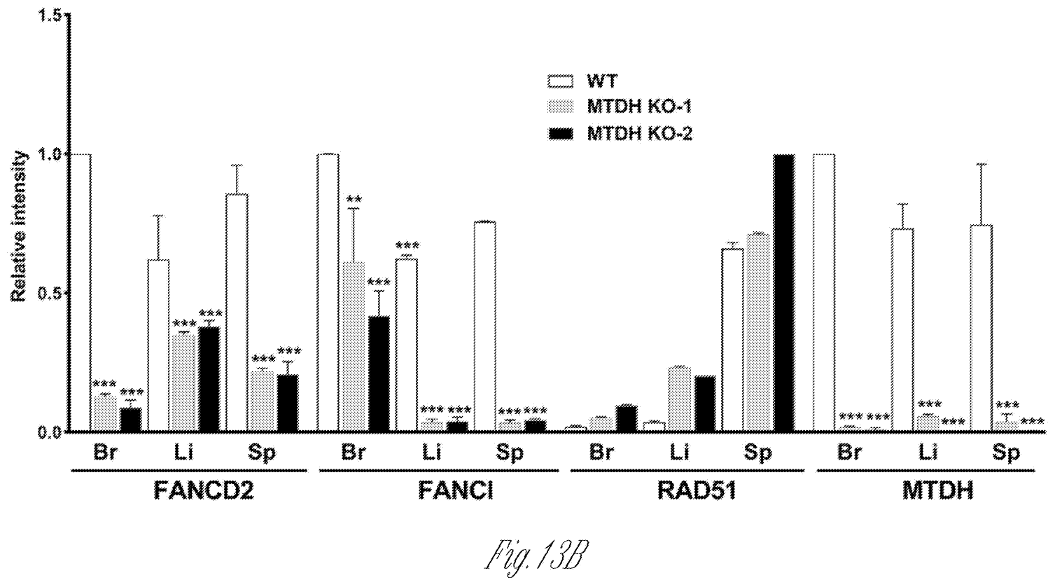

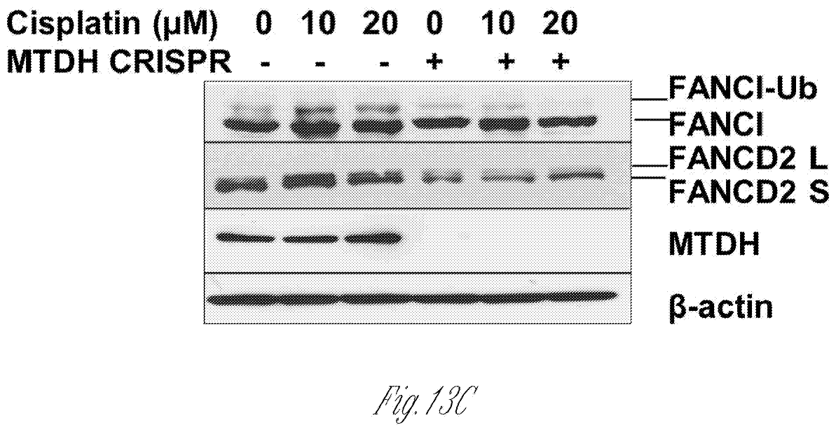

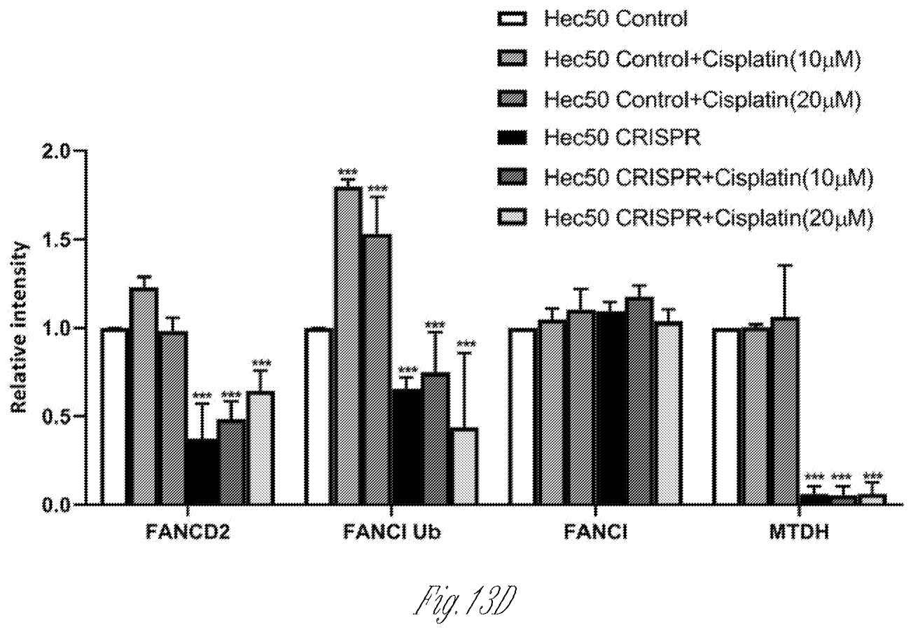

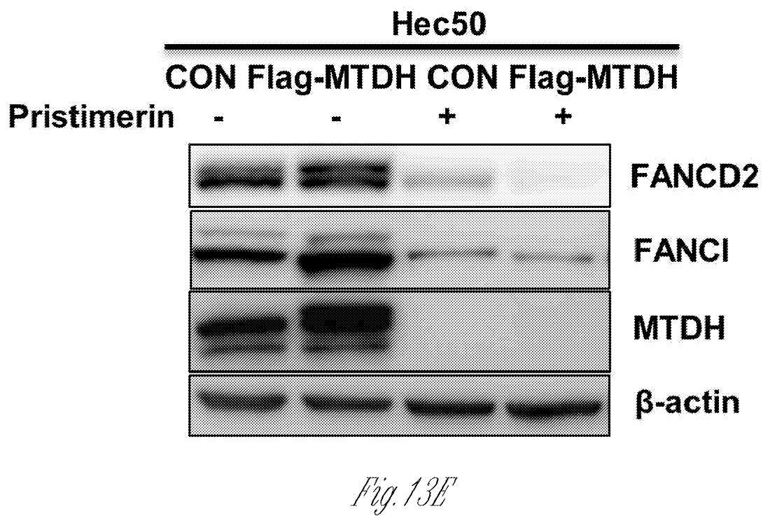

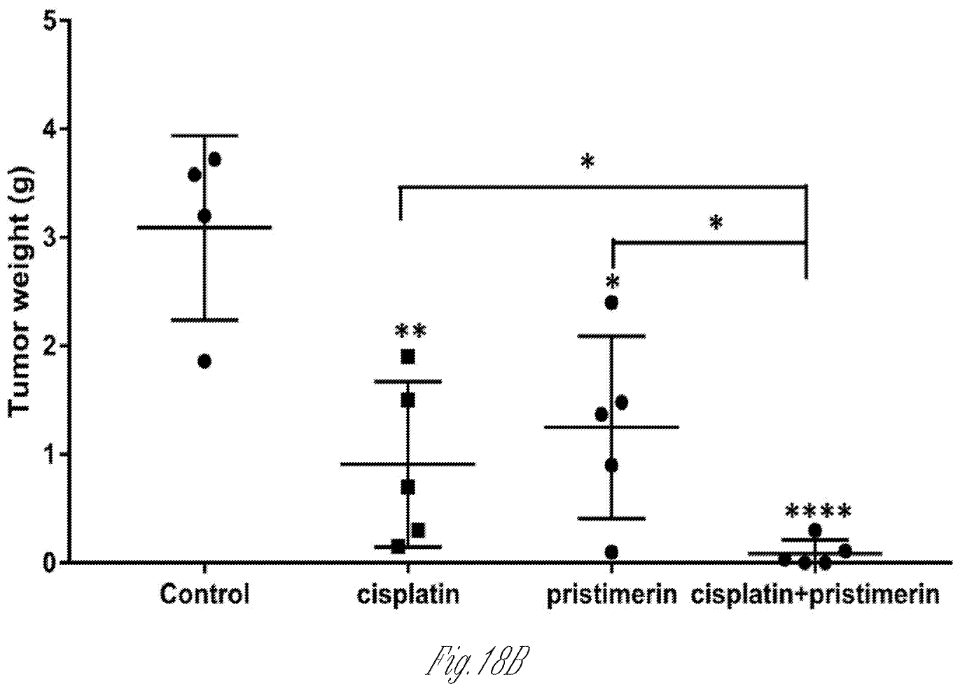

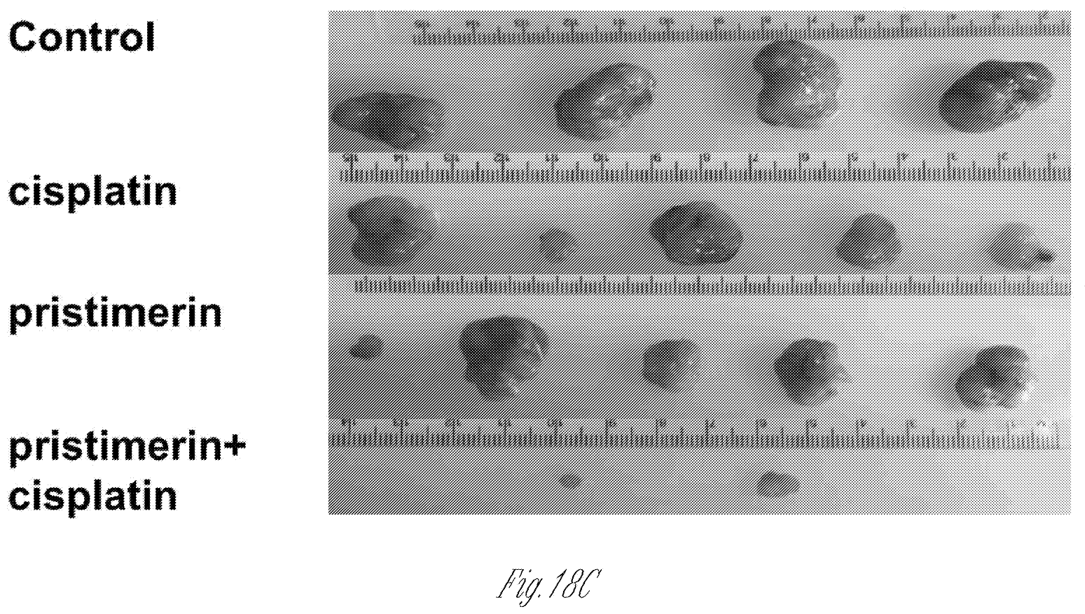

MTDH binds mRNAs corresponding to FANCD2 and FANCI proteins (Meng et al., 2012). This observation has been confirmed by analyzing FANCD2 and FANCI in GEO dataset GSE110260 by deep sequencing of MTDH-associated transcripts, which were precipitated by an anti-MTDH antibody after protein and mRNA crosslinking. Of note, several regions within the FANCI and FANCD2 mRNA sequences pulled-down by MTDH antibody were found in GSE110260 (Table 4). To determine whether MTDH contributes to changes in FANCD2 and FANCI expression at the protein level, expression of FANCD2, FANCI and other DNA repair proteins were examined in tissues from MTDH knockout mice, which were generated by homozygous deletion of exon 3 in the Mtdh gene. A dramatic reduction of FANCD2 and FANCI was detected in the liver, brain and spleen from MTDH knockout mice, though expression of other DNA repair proteins such as Rad51 remained unchanged (FIGS. 13A and 13B). Similarly, in endometrial cancer cells with genetic deletion of MTDH by CRISPR/Cas9 technology, we observed a marked reduction in FANCD2 protein expression as well as mono-ubiquitin conjugated FANCD2 in control and 10 μM and 20 μM cisplatin treated cells and a marked reduction of mono-ubiquitin conjugated FANCI in 10 μM and 20 μM cisplatin treated cells (FIGS. 13C and 13D). Monoubiquitin-conjugated FANCD2 and FANCI may be used as a biomarker to determine if the FA pathway is competent or deficient and to predict sensitivity to DNA crosslinking therapeutic agents (Ulrich & Walden, 2010). Reduction of monoubiquitin-conjugated FANCD2 and FANCI proteins indicates the reduction of activation of the FA repair pathway (Sims et al., 2007; Nepal et al., 2017). In endometrial cancer cells with MTDH overexpression, a marked increase in FANCD2 and FANCI and mono-ubiquitin conjugated FANCD2 and FANCI was observed (FIGS. 13E and 13F). By contrast, MTDH deletion and MTDH overexpression had no effect on mRNA levels on of FANCD2 and FANCI (FIG. 19), suggesting that the effect of MTDH on FA pathway protein expression is at the post-transcriptional level.

### Analysis of the Expression of MTDH and FANCI in Endometrial and Breast Cancer Patients

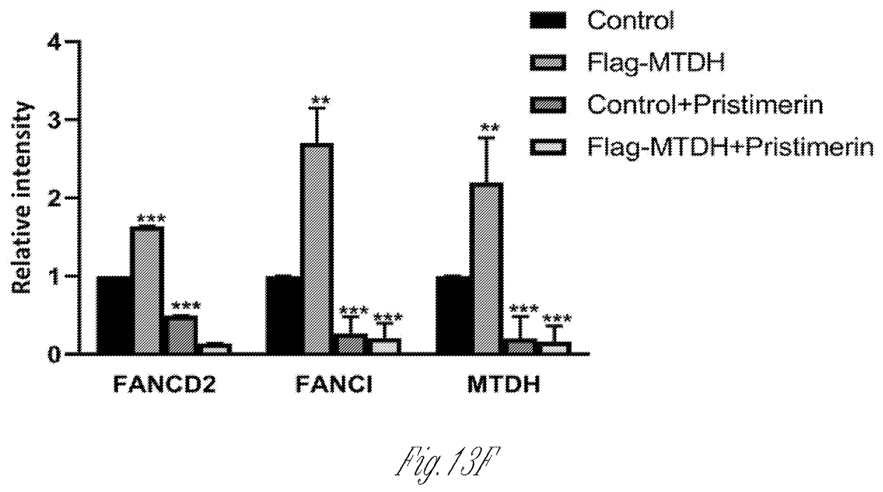

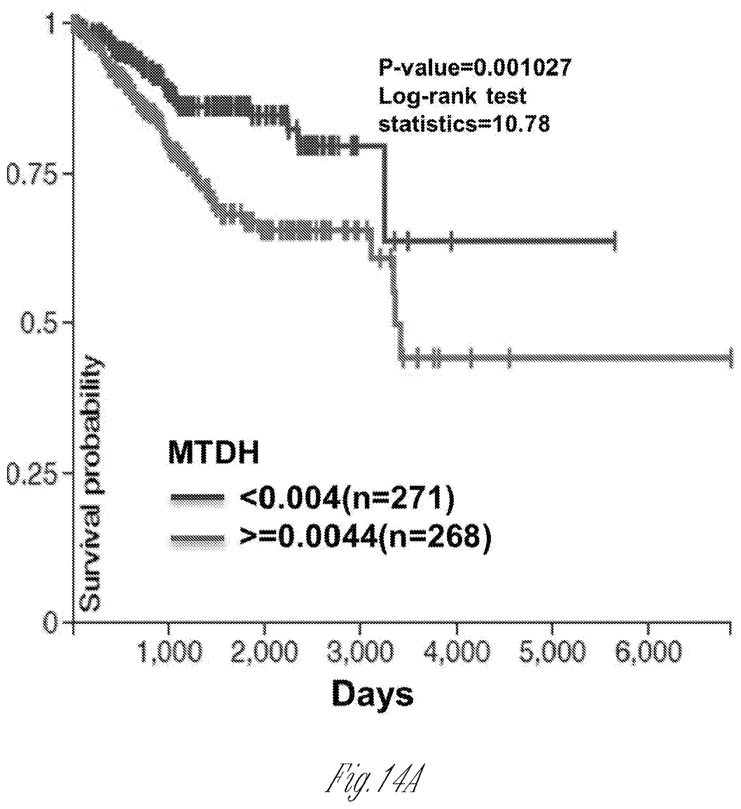

MTDH amplification negatively correlates with overall survival in breast cancer patients (Hu et al., 2009). Using TCGA dataset for endometrial cancer, it was substantiated that MTDH amplification is also associated with poor survival in endometrial cancer (FIG. 14A). To analyze whether MTDH expression is correlated with FANCD2 and FANCI in TCGA dataset, it was observed that amplification and increased expression of MTDH also positively correlated with the expression of FANCI and FANCD2 in the TCGA dataset for breast cancer (FIG. 14B). A positive correlation of MTDH with FANCD2 and FANCI at mRNA level was also observed in endometrioid endometrial cancer patients (Table 4).

TABLE 4
mRNA of MTDH positively correlated
with mRNAs of FANCD2 and
FANCI in Endometrioid Endometrial Cancer (EC).

| mRNA | mRNA | P_value | estimate | qFDR |
|---|---|---|---|---|
| MTDH | FANCD2 | 3.73E−27 | 0.59 | 5.60E−29 |
| MTDH | FANCI | 6.69E−20 | 0.52 | 1.24E−21 |

Identification of the Region in MTDH that Associates with FANCD2 and FANCI mRNAs

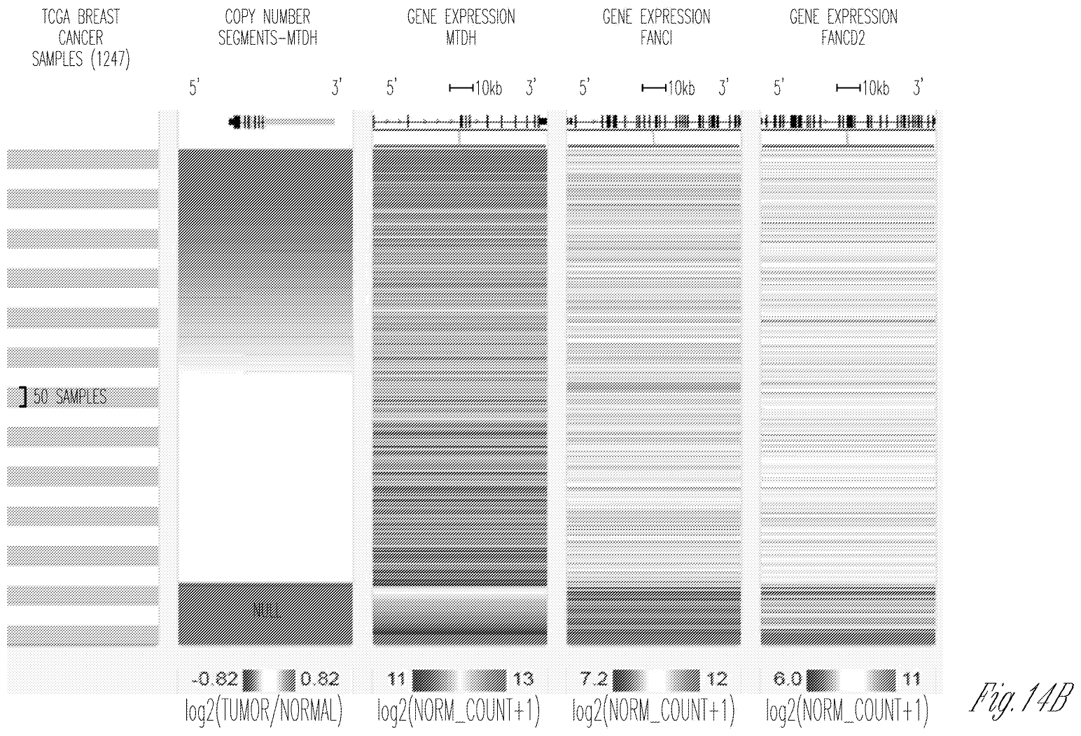

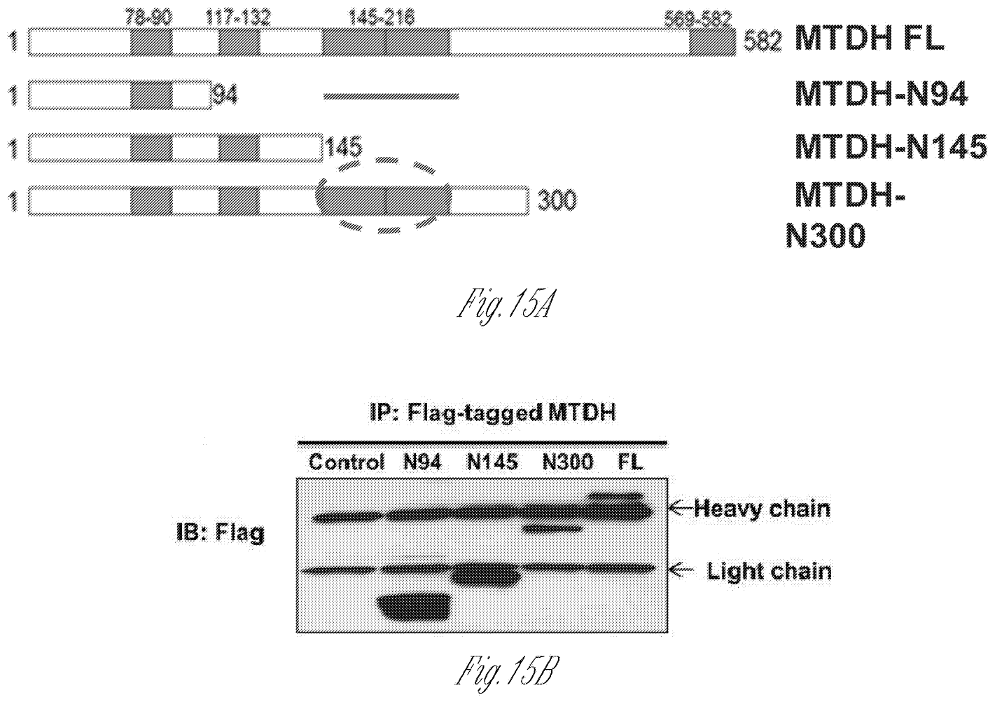

A previous study showed four putative RNA binding regions in MTDH (Meng et al., 2012). To identify the specific region in MTDH that binds mRNAs, FLAG-tagged fragments of MTDH were transiently expressed in Hec50 cells in which endogenous MTDH was knocked down (FIGS. 15A, 15B). Protein extracts were subjected to anti-FLAG antibody pull-down followed by RT-qPCR to detect MTDH-bound FANCI and FANCD2 mRNAs. It was found that residues 145-216 were essential for the association of MTDH with FANCD2 and FANCI mRNAs (FIG. 15C).

### MTDH Silencing Increases α-H2AX Foci Formation and Sensitivity to Cisplatin in Cancer Cells

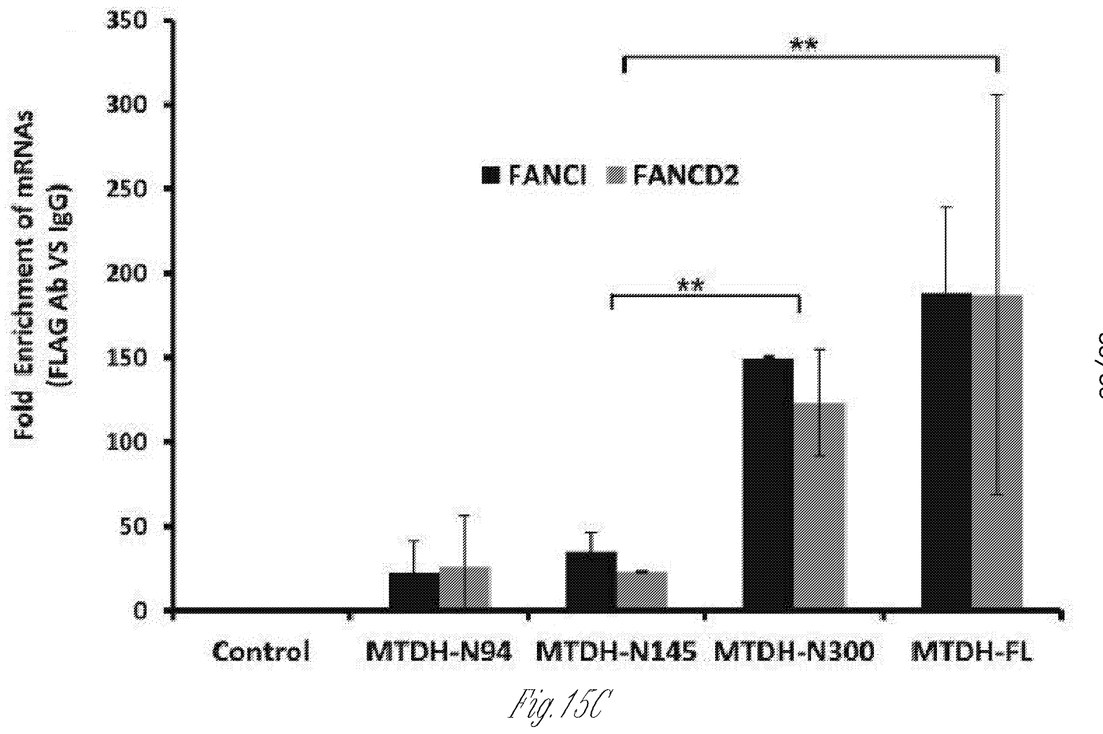

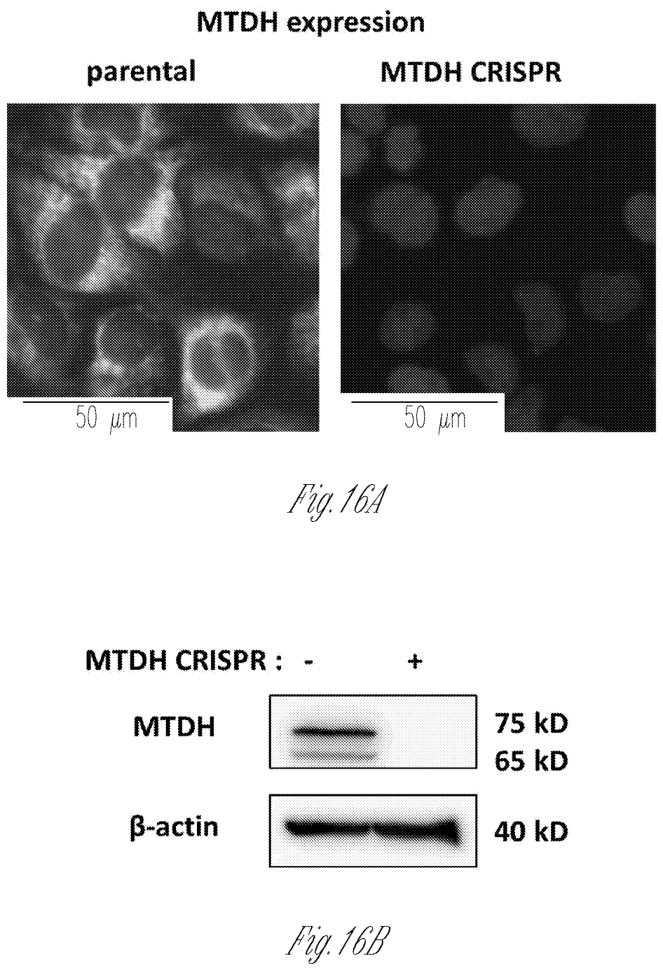

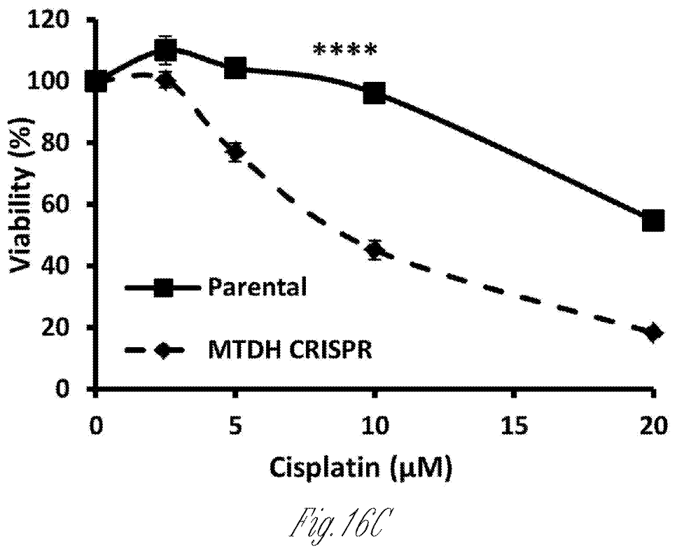

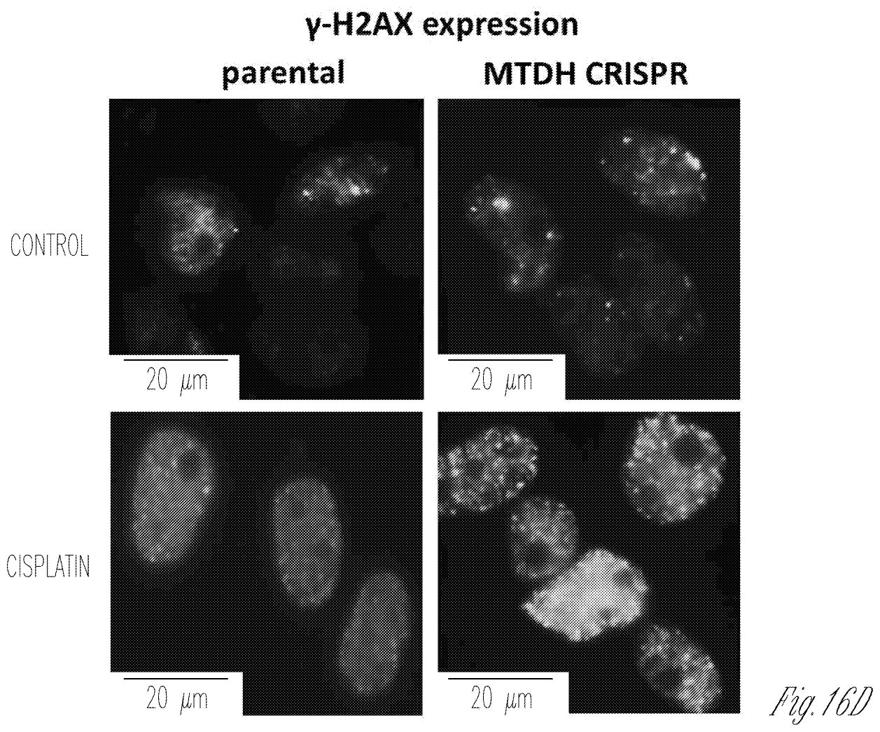

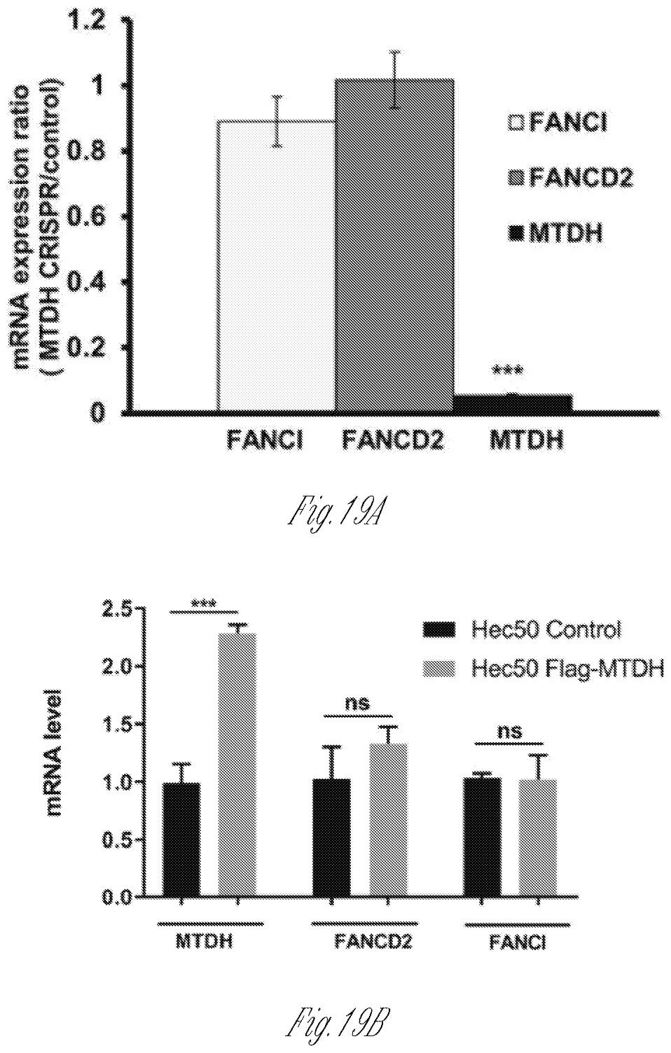

The FA pathway plays a critical role in the repair of DNA crosslink damage induced by chemotherapeutic agents including cisplatin (Kim & D'Andrea, 2012). Consistent with previous reports (Meng et al., 2012), MTDH deficiency significantly increased sensitivity to the DNA damaging agent cisplatin (FIGS. 16A-16C). No difference of cell proliferation between cancer cells expressing scrambled sgRNA and cancer cells with multiple MTDH knockout clones by expressing MTDH sgRNA (FIG. 20). The impact of MTDH on DNA damage repair was directly tested by assessing α-H2AX foci formation, a standard biomarker to denote an increase in DNA damage (Nowsheen et al., 2009). Formation of cisplatin-induced α-H2AX foci was significantly increased in MTDH-deficient Hec50 cells (FIGS. 16D, 16E). From these data, it was concluded that MTDH is required to repair cisplatin induced DNA damage.

### Pristimerin Increases Cisplatin Sensitivity by Downregulating MTDH

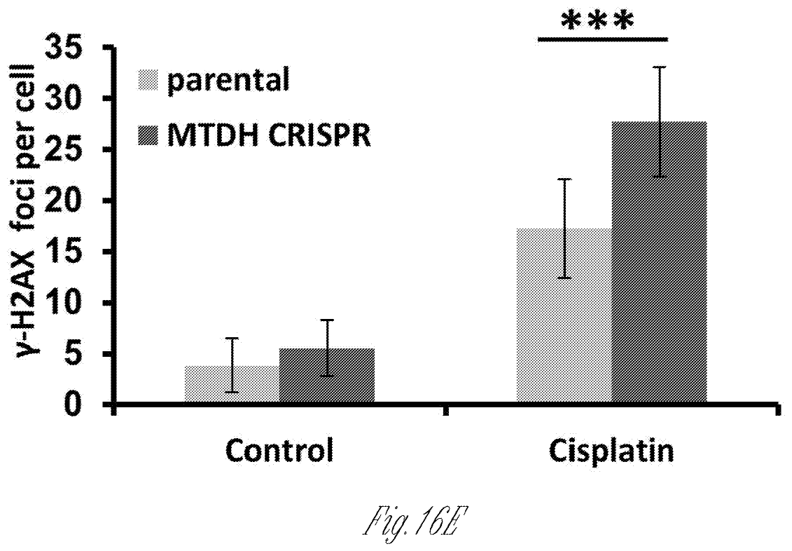

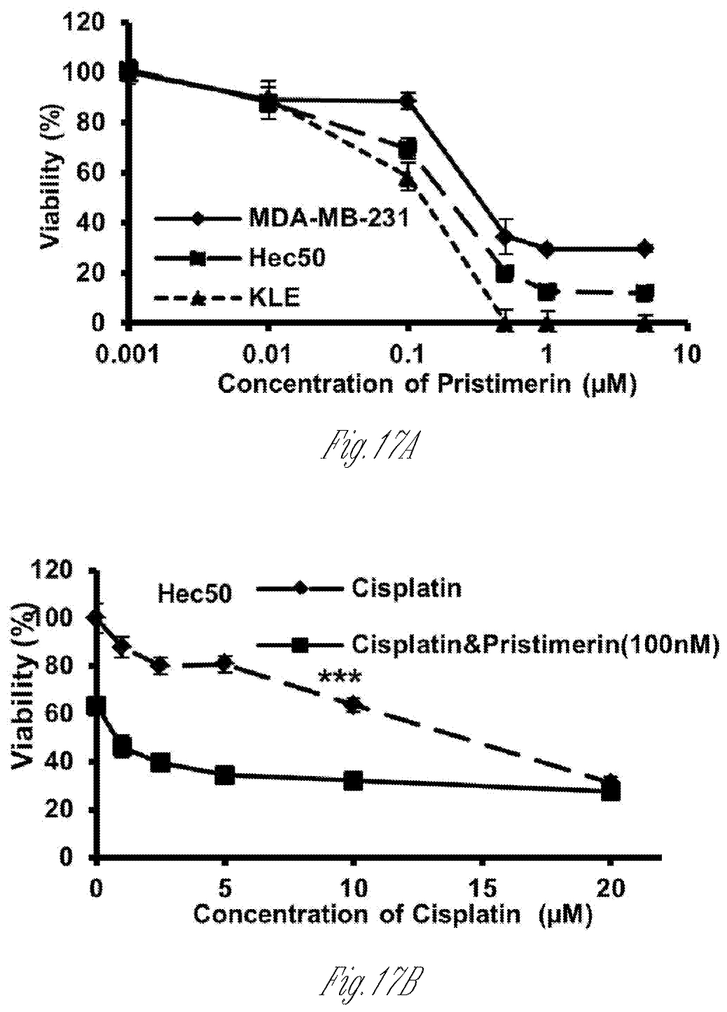

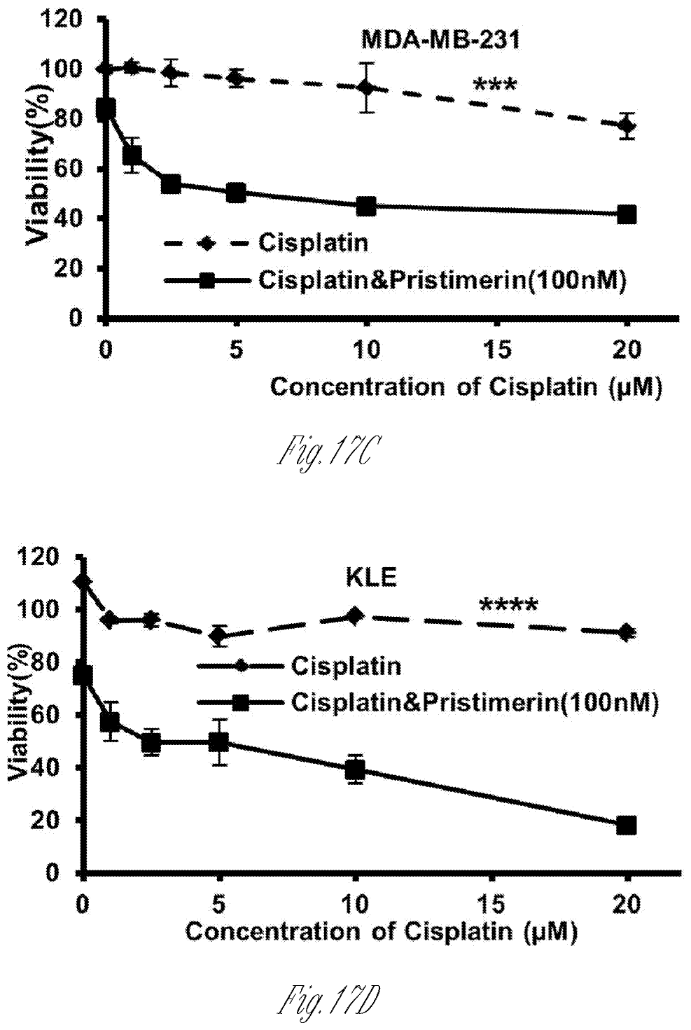

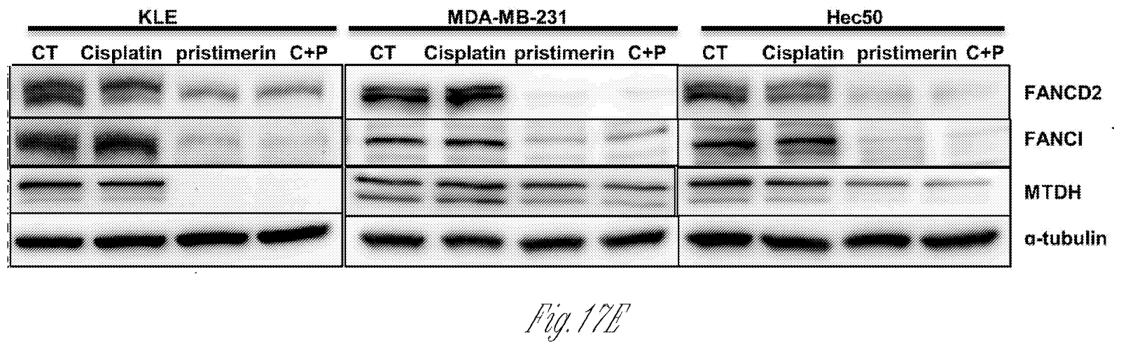

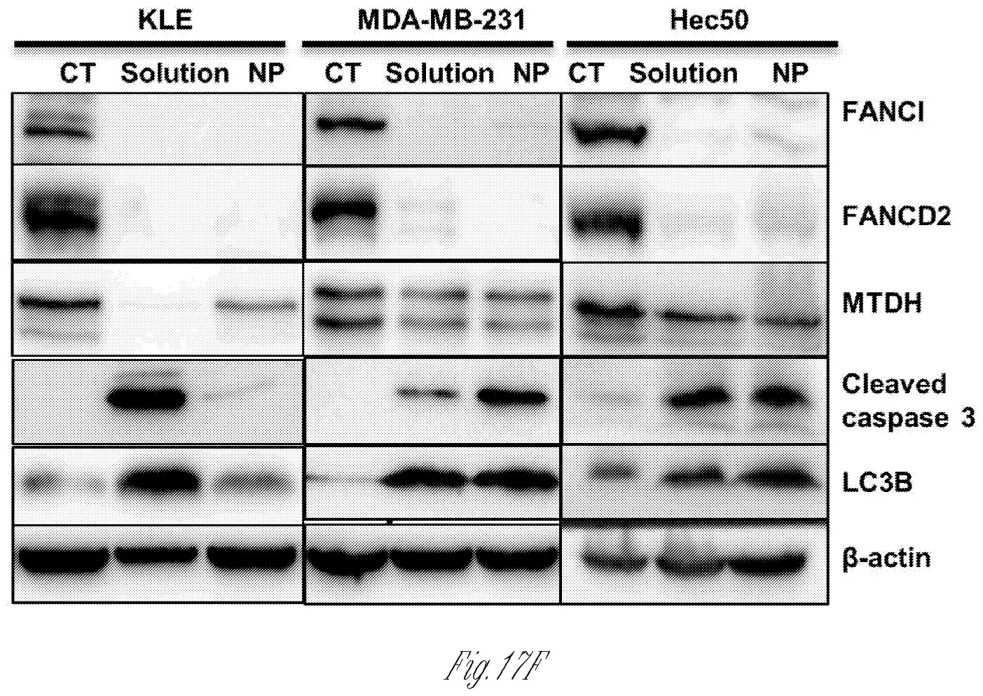

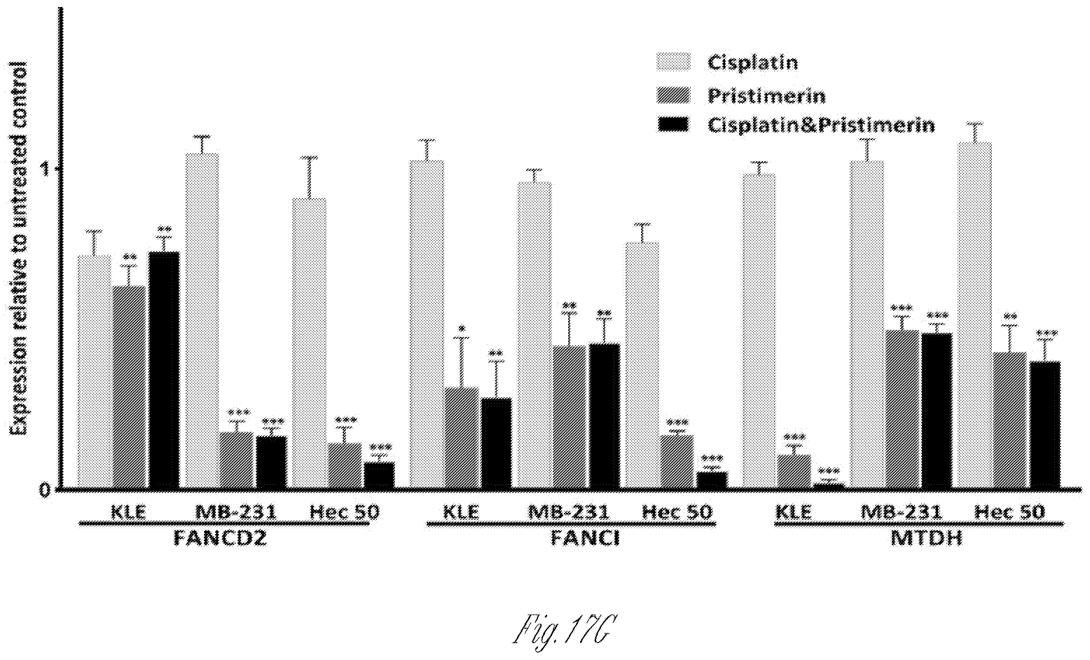

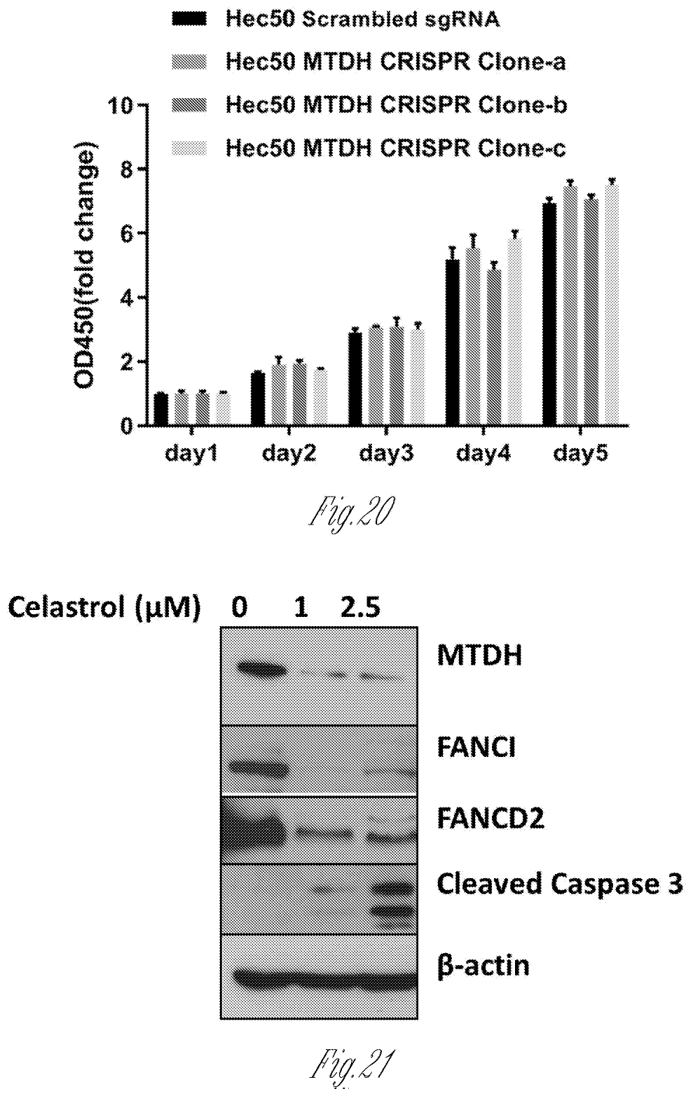

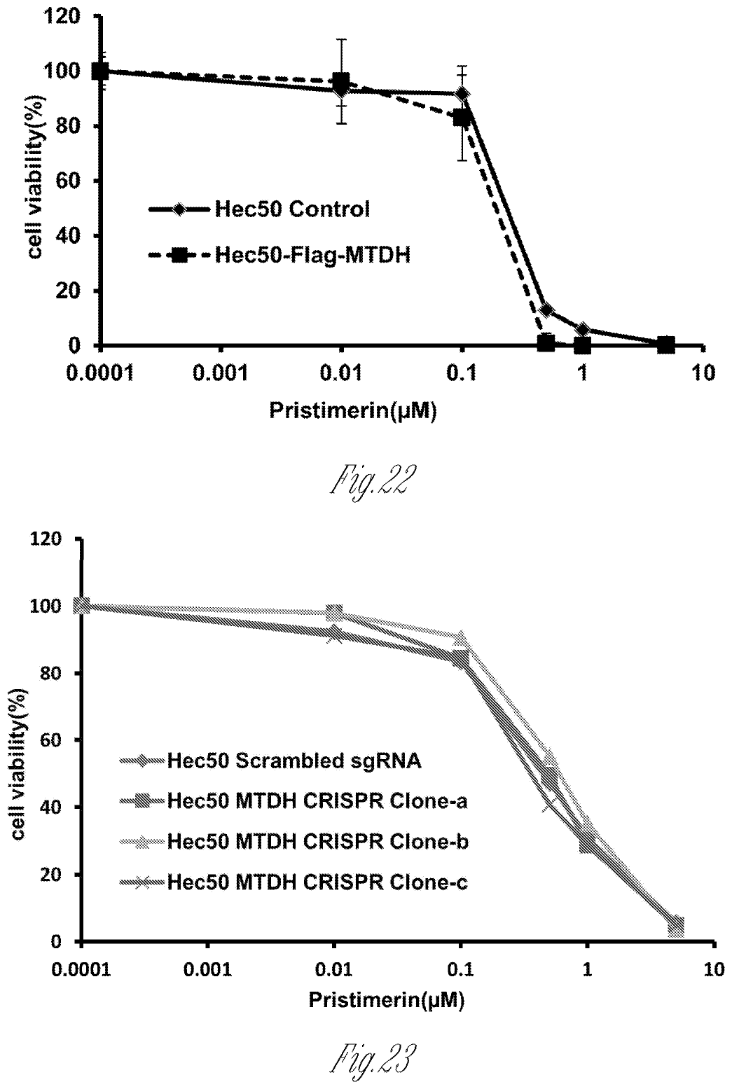

Directly targeting MTDH through genetic manipulation is not currently feasible in patients. Therefore, small molecules that can decrease MTDH expression were identified. A recent study in lung cancer cells found that celastrol, a natural agent, promotes proteasomal degradation of FANCD2, thereby increasing sensitivity to DNA crosslinking agents (Wang et al., 2015). It was found that celastrol can also reduce MTDH and FANCI protein levels in cancer cells (FIG. 21). However, celastrol is a leptin sensitizer and leads to weight loss in obese mice (Liu et al., 2015). To avoid weight loss in cancer patients, another compound with a similar quinonemethide triterpenoid structure, pristimerin, which has shown promising in tumor growth inhibition in preclinical study, was tested (Yousef et al., 2017). It was first established that pristimerin decreases viability of Hec50, MDAMB-231 and KLE cells, with IC50 values below 1 μM (FIG. 17A). Overexpression of MTDH was not protective of pristimerin-induced cell death (FIG. 22). No change of sensitivity to pristimerin was observed in scrambled sgRNA or multiple clones with MTDH depletion by sgRNA against MTDH (FIG. 23). At doses as low as 100 nM, pristimerin increased sensitivity to cisplatin in all three cancer cell lines (FIGS. 17B, 17D). Importantly, pristimerin (in solution) decreased MTDH, FANCD2 and FANCI protein levels when used as a single drug or in combination with cisplatin in all three tested cell lines (FIG. 17E). Overexpression of MTDH does not inhibit pristimerin-induced decrease of MTDH, FANCD2 and FANCI (FIGS. 13E and 13F). These data demonstrate that treatment with pristimerin is a potential therapeutic approach to overcome the effects of high MTDH expression.

### Quantification and Characterization of Pristimerin-Loaded PLGA Nanoparticles

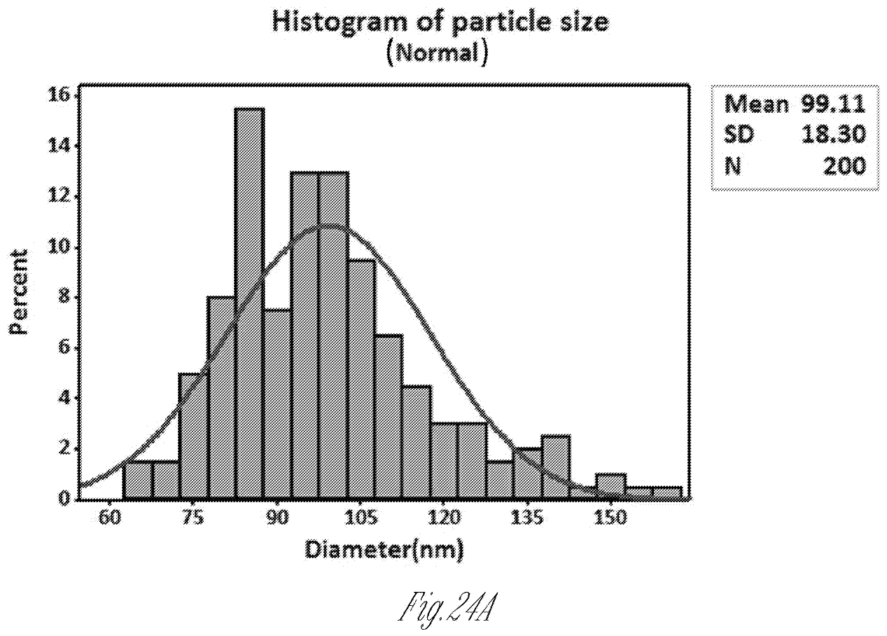

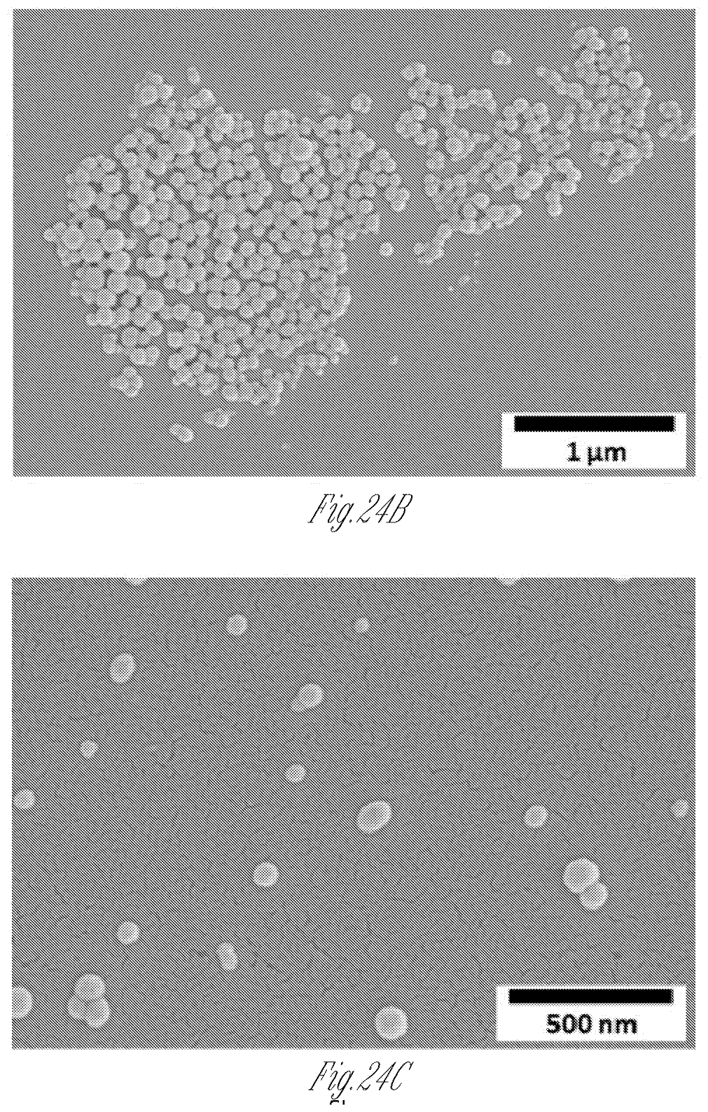

Similar to previous reported poor solubility and pharmacokinetics of celastrol (Guo et al., 2017), pristimerin in solution did not induce significant tumor growth inhibition in a preliminary study in PDX mouse model of endometrial cancer (data not shown). A nanoparticle-based delivery approach was used to improve the pharmacokinetics and therapeutic efficacy of pristimerin. Pristimerin was loaded into PLGA nanoparticles. The amount of pristimerin-loaded nanoparticles was quantified by HPLC. The drug loading and encapsulation efficiency of pristimerin were 168.70±40.56 mg/mg and 101.22±24.38%, respectively. Particles were characterized by scanning electron microscopy (SEM), which demonstrated that the particle morphology is spherical with a smooth surface. The average particle size was 99.11±18.30 nm (FIG. 24A). The zeta potential measured by the dynamic light scattering method was −46.82±6.64 mV (FIG. 24B).

### Nanoparticle-Delivered Pristimerin Inhibits MTDH, FANCD2 and FANCI in Cancer Cells

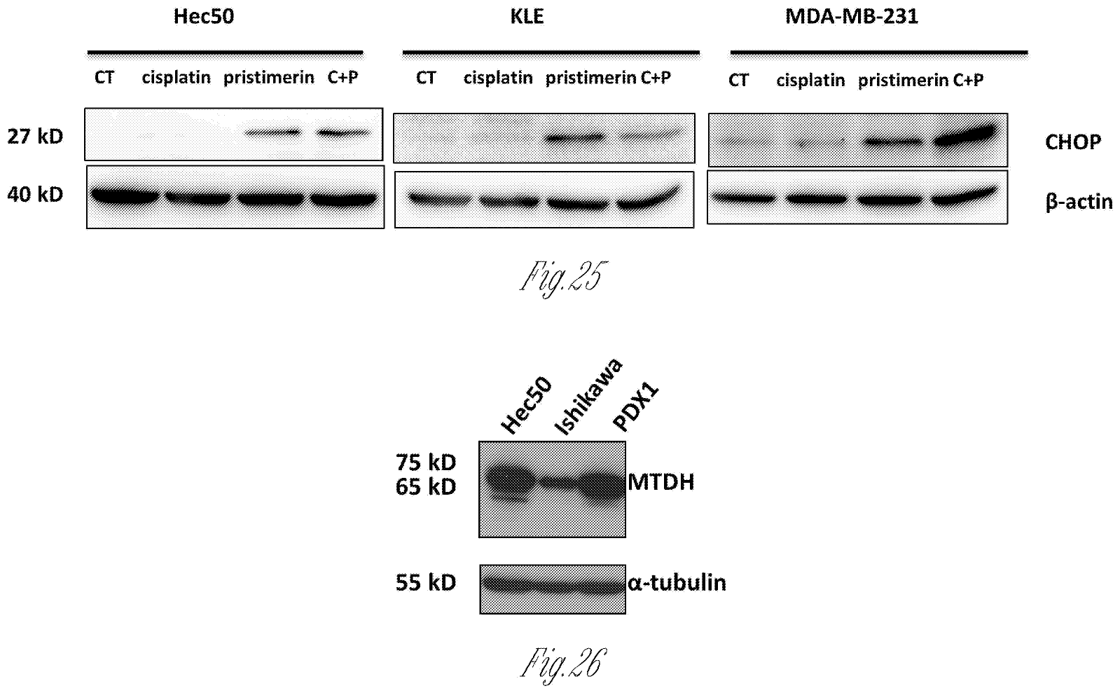

It was established that pristimerin-loaded nanoparticles reduced protein expression of MTDH, FANCD2 and FANCI to levels similar to those achieved using pristimerin in solution in cell models (FIG. 17F). In addition, protein levels of the endoplasmic reticulum (ER) stress biomarker CHOP, the apoptosis biomarker cleaved caspase 3 and the autophagy biomarker LC3B were all increased by treatment of Hec50, MDA-MB-231 and KLE cells with pristimerin in solution and pristimerin-loaded nanoparticles (FIG. 17F and FIG. 25). Pristimerin-loaded nanoparticles induce similar level of cleaved caspase 3, LC3B and CHOP expression compared to the pristimerin in solution in MDA-MB-231 and Hec50 cells, but less in KLE cells. These data substantiate the efficacy of nanoparticle-delivered pristimerin in downregulating MTDH as well as implicating the involvement of ER stress, apoptosis and autophagy in the mechanism of cell death in response to pristimerin.

Cisplatin Combined with Pristimerin Inhibits Tumor Growth in a Patient-Derived Xenograft Mouse Model

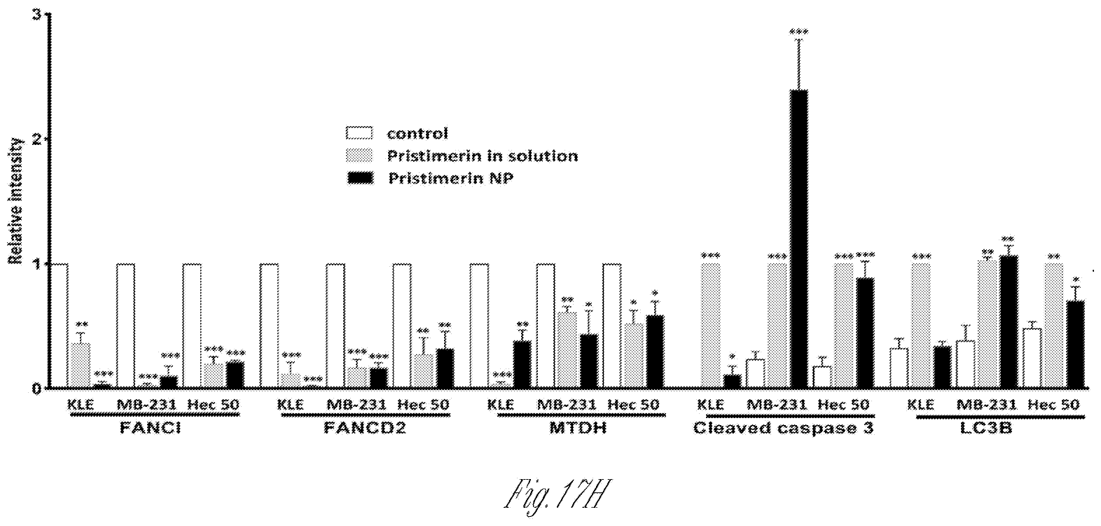

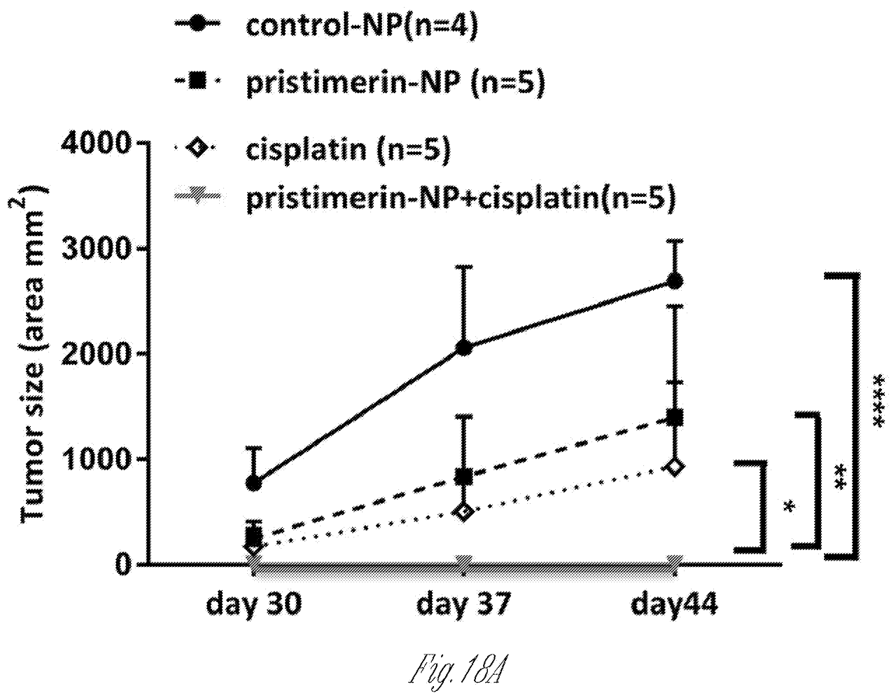

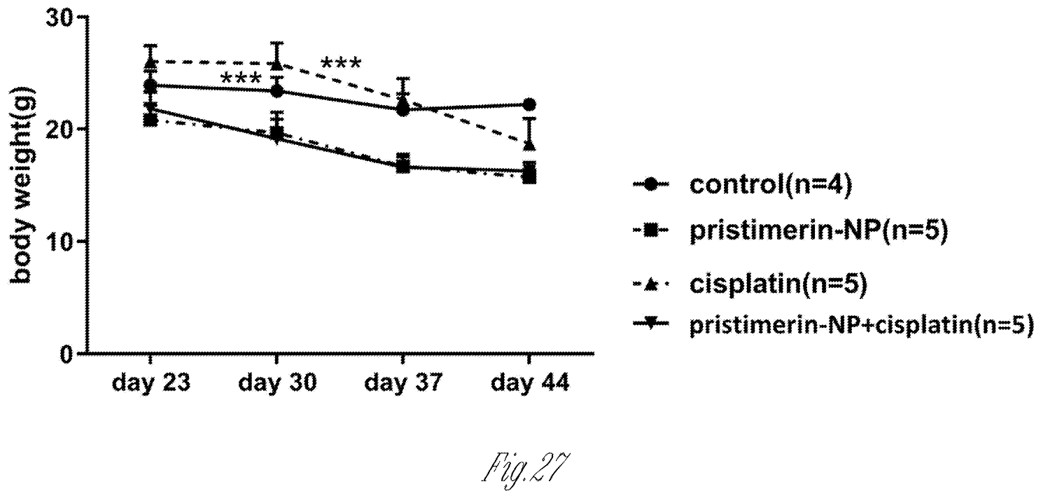

To investigate the effects of pristimerin on tumor growth, studies were performed in a PDX model of serous endometrial cancer. This model, denoted PDX1 herein, was previously developed by implanting a fresh surgically resected endometrial tumor specimen into the subcutis of immunocompromised mice (Luo et al., 2010). PDX1 tumors are subsequently passaged in mice. High expression of MTDH in this model was confirmed, with levels similar to those observed in Hec50 cells (FIG. 26). Next, immunocompromised mice bearing PDX1 tumors were divided into four different treatment groups: control (empty) PLGA nanoparticles, cisplatin, nanoparticle-loaded pristimerin and the combination of cisplatin with pristimerin-loaded nanoparticles. Treatment with cisplatin or pristimerin alone significantly inhibited tumor growth as compared to control PLGA nanoparticles (P<0.05). However, the combination of cisplatin and nanoparticle-loaded pristimerin further decreased the tumor growth (P<0.001 compared to all other groups, FIG. 6A), with a corresponding reduction in tumor weight at 30 days after treatment (P<0.001) (FIGS. 18B, 18C). Similar to celastrol, pristimerin also caused weight loss in mice treated with nanoparticle-loaded pristimerin alone or in combination with cisplatin (FIG. 27). These data validate pristimerin-loaded nanoparticles as a potential treatment to restore sensitivity to cisplatin in tumors with MTDH upregulation.

### Discussion

Platinum compounds are some of the most effective broad-spectrum anti-cancer chemotherapeutic drugs (Desoize & Madoulet, 2002). They function by inducing DNA cross-linking damage in cancer cells in a wide range of cancer types. Unfortunately, drug resistance occurs gradually and frequently in patients whose tumors were initially sensitive to platinum agents (Muggia, 2004). One mechanism of resistance is an increased ability of cancer cells to repair platinum-induced DNA damage (Tortorella et al., 2018). DNA interstrand-crosslink damage is mainly recognized by proteins in the FA pathway and subsequently repaired by the homologous recombination repair (HRR) pathway (Ceccaldi et al., 2016). The majority of studies of FA-mediated DNA repair in cancer focus on inactivating mutations in FA genes. Indeed, the 17 FA genes in the FA pathway are frequently mutated across 68 DNA sequence datasets of non-Fanconi Anemia human cancers, at a rate in the range of 15 to 35% (Shen et al., 2015). BRCA2 is among these 17 genes, and studies in ovarian cancer demonstrate that tumors with mutations in BRCA2 are initially sensitive to platinum compounds due to loss of DNA repair capabilities (Sakai et al., 2009).

This canonical DNA repair mechanism can also be co-opted to drive chemoresistance, as disclosed herein. Specifically, overexpression of MTDH up regulates FANCD2 and FANCI by interacting with and promoting translation of FANCD2 and FANCI mRNAs. By upregulating these DNA repair proteins, MTDH induces significant resistance to DNA-damaging agents by endowing cancer cells with an enhanced ability to repair damaged DNA. Consistent with these findings, others have found that FANCD2 expression is up regulated and correlates with poor outcome in hepatocellular carcinoma (Komatsu et al., 2017). Despite a loss of protein expression, no changes in mRNA levels of FANCD2 or FANCI were detected in MTDH-deficient cells. Hence, MTDH regulates FA family proteins at the post-transcriptional level. Consistent with this interpretation, MTDH has been found to bind to several target sequences in the coding region and 3-terminal untranslated region of FANCI (Table 5 herein) (Hsu et al., 2018)).

TABLE 5
FANCI and FANCD2 mRNAs sequences pulled-down by PAR-CLIP
(Photoactivatable Ribonucleoside-Enhanced Crosslinking and
Immunoprecipitation) anti-MTDH antibody derived from GEO dataset
GSE110260.

| | | | | | |
|---|---|---|---|---|---|
| NM_018193 | FANCI | 68 | 123 | 7.648886902 | CDS(the coding |
| | | 924 | 955 | 5.979785428 | sequences) |
| | | 1023 | 1068 | 7.063917906 | From 91 to 3897 |
| | | 1349 | 1508 | 8.648887138 | |
| | | 1916 | 2098 | 7.063924637 | |
| | | 2206 | 2272 | 7,648886902 | |
| | | 2364 | 2409 | 6.979785428 | |
| | | 3194 | 3258 | 6.063924637 | |
| | | 3370 | 3413 | 7.063917906 | |
| | | 3507 | 3610 | 6.063924401 | |
| | | 4274 | 4356 | 6.063917906 | |
| | | 4360 | 4443 | 6.059907037 | |
| NM_001018115 | FANCDI | 1353 | 1416 | 9.242527026 | CDS |
| | | 4360 | 4410 | 8.162405417 | From 121 |
| | | 4442 | 4499 | 9.162405417 | to 4476 |

Since MTDH regulates the expression of a cadre of FA pathway factors through its novel RNA binding properties, it was hypothesized that MTDH would be a good therapeutic target by which to increase sensitivity to platinum compounds. Currently, no MTDH specific inhibitors are available due to the lack of canonical catalytic domains in MTDH. The discovery that pristimerin can efficiently reduce expression of MTDH and FA pathway proteins provides a potential solution to repurpose this anti-inflammatory drug as a novel agent to combine with chemotherapy. Pristimerin is a natural triterpenoid isolated from the Celastraceae and Hippocrateaceae plant families and is widely used in traditional Chinese medicine as an anti-inflammatory medication (Tong et al., 2014). Multiple preclinical studies in a wide range of cancer types, including breast cancer, colon cancer, prostate cancer and pancreatic cancer, confirm the antitumor activity of pristimerin (Yousef et al., 2017). Mechanistic studies have suggested that the anti-inflammatory activity of pristimerin is accomplished through inhibition of the well-known pro-inflammatory transcription factor NF-κB via inhibition of the NF-κB inhibitor IKK (Hui et al., 2014). In addition, pristimerin has been shown to inhibit chymotrypsin-like protease activity (Tiedemann et al., 2009), suggesting that pristimerin is a dual proteasome and NF-κB inhibitor. Of note, NF-κB regulates expression of MTDH by binding to the promoter of the MTDH gene (Sakar et al., 2008). Therefore, we speculate that pristimerin accomplishes the reduction of MTDH expression by interfering with NF-κB-mediated transcription of this gene.

To enhance drug solubility, stability and accumulation in the tumor, a nanoparticle formulation was used to deliver pristimerin to tumors in vivo. Nanoparticles have been extensively utilized for delivering therapeutic and diagnostic agents. Nanoparticles offer a superior dissolution profile of their payload due to their unique size range that governs a vast increase in the exposed surface area to the dissolution medium (Kelidari et al., 2017). Nanoparticles prepared from natural or synthetic polymers modify drug release and create a sustained or controlled release profile (Breitenbach et al., 2000). The specific nanoparticle formulation used to deliver pristimerin, which consists of PLGA at a monomer ratio of 75:25 and TPGS surfactant, improves therapeutic efficacy of pristimerin through enhanced drug uptake and accumulation. TPGS has a unique ability to inhibit P-glycoprotein (P-gp) efflux transporter, a transporter that is highly overexpressed in many cancers (Duhem et al., 2014; Collnot et al., 2007), which can extrude drug substances out of the cells, reducing their intracellular concentration and effect. Many studies indicated that pristimerin is a substrate to P-gp, therefore loading pristimerin in NPs containing TPGS would enhance its intracellular accumulation through inhibiting its efflux (Zhao et al., 2018). Loading a substrate to P-gp efflux transporter in NPs prepared with TPGS significantly improved the substrates intracellular accumulation once compared to its soluble form (Ebeid et al., 2018). Regarding cancer treatment, nanoparticles <200 nm in diameter offer superior accumulation at the tumor site due to the enhanced permeability and retention (EPR) effect.

In conclusion, the present data support the role of MTDH overexpression as a mechanism that leads to resistance to chemotherapy via its novel RNA binding function. It was also demonstrated that inhibition of MTDH expression leads to a significant reduction in FA DNA repair proteins, and this effect can be phenocopied by treating with pristimerin-loaded nanoparticles. Thus, the present data provide a foundation for these nanoparticles as a therapeutic strategy to improve chemosensitivity.

### REFERENCES

Acharya & Sahoo, Advanced Drug Delivery Reviews, 63:170 (2011).
Brabec & Kasparkova, Drug Resistance Updates: Reviews and Commentaries in Antimicrobial and Anticancer Chemotherapy, 8:131 (2005).
Burger et al., Drug Resistance Updates: Reviews and Commentaries in Antimicrobial and Anticancer Chemotherapy, 14:22 (2011).
Ceccaldi et al., Nature Reviews Molecular Cell Biology, 17:337 (2016).
Cheng et al., Biomaterials, 28:869 (2007).
Deans & West, Nature Reviews Cancer, 11:467 (2011).
Desoize & Madoulet, Critical Reviews in Oncology/Hematology, 42:317 (2002).
Ebeid et al., Nature Nanotechnology, 13:72 (2018).
El Botty et al., Oncotarget, 9:29587 (2018).
Emdad et al., Advances in Cancer Research, 131:97 (2016).
Emdad et al., Cancer Research, 66:1509 (2006).
Galluzzi et al., Oncogene, 31:1869 (2012).
Gravanis & Gurpide, Journal of Steroid Biochemistry, 24:469 (1986).
Hellweg et al., Methods in Molecular Biology (Clifton, N.J.), 1907:83
(2019).
Hsu et al., RNA (New York, N.Y.), 24:688 (2018).
Hu et al., Cancer Cell, 15:9 (2009).
Hui et al., International Immunopharmacology, 21:501 (2014).
Kavlashvili et al., PloS One, 11:e0148912 (2016).
Kim & D'Andrea, Genes & Development, 26:1393 (2012).
Komatsu et al., Anticancer Research, 37:1083 (2017).
Lee et al., Proceedings of the National Academy of Sciences of the United States of America, 103:17390 (2006).
Luo et al., International Journal of Oncology, 37:1187 (2010).
Meng et al., Advances in Cancer Research, 120:135 (2013).
Meng et al., The Journal of Biological Chemistry, 287:4485 (2012).
Meng et al., The Journal of Biological Chemistry, 290:11853 (2015).
Moelans et al., Cancer Letters, 344:138 (2014).
Muggia, Seminars in Oncology, 31:17 (2004).
Nakanishi et al., Proceedings of the National Academy of Sciences of the United States of America, 102:1110 (2005).
Nowsheen et al., Mutation Research, 674:131 (2009).
Park et al., Phytomedicine: International Journal of Phytotherapy and Phytopharmacology, 42:164 (2018).
Sakai et al., Cancer Research, 69:6381 (2009).
Sakai et al., Nature, 451:1116 (2008).
Sarkar et al., Cancer Research, 68:1478 (2008).
Shen et al., Cancer Research, 73:3393 (2013).
Shen et al., Oncotarget, 6:20396 (2015).
Shkreta & Chabot, Biomolecules, 5:2935 (2015).
Song et al., PloS One, 10:e0127599 (2015).
Taniguchi et al., Cell, 109:459 (2002).
Thompson, Nature Genetics, 37:921 (2005).
Tiedemann et al., Blood, 113:4027 (2009).
Tortorella et al., Gynecologic Oncology, 149:291 (2018).
van der Heijden et al., Cancer Research, 63:2585 (2003).
Venkitaraman, Nature Reviews Cancer, 4:266 (2004).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising nanoparticles comprising an amount of one or more quinonemethide triterpenoids effective to enhance sensitivity to platinum compounds.

2. A composition comprising nanoparticles comprising an amount of one or more inhibitors of metadherin effective to enhance sensitivity to platinum compounds.

3. The composition of claim 1 wherein the diameter of the nanoparticle is 200 nm or less.

4. The composition of claim 1 wherein the quinonemethide triterpenoid or the inhibitor comprises pristimerin, celastrol, lupeol, hydroxy-pristimerin, Tingenin B, tripterin, tripterygone, or 2-acetylphenol-1-beta-D-glucopyranosyl (1-->6)-beta-D-xylpyranoside.

5. The composition of claim 1 wherein the nanoparticle has a diameter of about 25 nm to about 200 nm or about 75 nm to about 110 nm.

6. The composition of claim 1 wherein the nanoparticle comprises a synthetic polymer comprising lactic acid, glycolic acid, caproic acid, a polyanhydride, PEI, or a combination thereof.

7. (canceled)

8. The composition of claim 6 wherein the polymer comprises lactic acid and glycolic acid, polycaprolactone or polylactic acid.

9. The composition of claim 6 wherein the polymer comprises a ratio of lactic acid to glycolic acid of 70:30, 75:25, 80:20, 65:35, 60:40, 55:45 or 50:50.

10. (canceled)

11. The composition of claim 1 wherein the nanoparticle further comprises a targeting ligand.

12. A method to enhance sensitivity to an antineoplastic agent in a mammal having cancer, comprising: administering to the mammal the composition of claim 1.

13. The method of claim 12 wherein the mammal is a human.

14. The method of claim 12 wherein the cancer is testicular cancer, ovarian cancer, lung cancer, lymphoma, bladder cancer, cervical cancer, breast cancer, esophageal cancer, colon cancer, mesothelioma, pancreatic cancer, prostate cancer, brain cancer, neuroblastoma, endometrial cancer, small cell lung cancer, ovarian cancer, triple negative breast cancer or head and neck cancer.

15. (canceled)

16. The method of claim 12 further comprising administering an antineoplastic agent.

17. The method of claim 16 wherein the antineoplastic agent cross links DNA, inhibits DNA repair, inhibits DNA synthesis, or a combination thereof.

18. The method of claim 16 wherein the agent is a platinum compound.

19-22. (canceled)

23. The method of claim 12 wherein the composition is locally administered.

24. The method of claim 12 wherein the composition is systemically administered.

25. The method of claim 12 wherein the composition is orally administered or intravenously administered.

26. (canceled)

27. The method of claim 12 wherein the cancer, prior to administration of the composition, is resistant to an antineoplastic agent.

28. The method of claim 27 wherein prior to administration of the composition, the cancer is resistant to an antineoplastic agent that cross links DNA, inhibits DNA repair, inhibits DNA synthesis, or a combination thereof.

29-32. (canceled)
